(19) United States
(12) Patent Application Publication
(10) Pub. No.: US 2009/0047287 A1
(43) Pub. Date: Feb. 19, 2009

(54) MODULATION OF BONE FORMATION

(75) Inventors: Julia Billiard, Collegeville, PA (US); Yan Liu, Collegeville, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/278,710

(22) PCT Filed: Feb. 16, 2007
(86) PCT No.: PCT/US07/04510

§ 371 Date: Aug. 7, 2008

Related U.S. Application Data
(60) Provisional application No. 60/774,534, filed on Feb. 17, 2006.
(60) Provisional application No. 60/844,239, filed on Sep. 13, 2006.

(51) Int. Cl. A61K 39/395; C12N 5/08; C12Q 1/48; C12Q 1/68; C07K 16/22; A61K 31/7105; C07K 14/51; A61P 19/08

(52) U.S. Cl. 424/139.1; 435/377; 435/15; 435/6; 530/387.9; 514/44; 530/350

(57) ABSTRACT

This invention relates to modulating Ror activity (e.g., Ror2 protein activity) and/or 14-3-3 β to affect bone formation or resorption. The invention further relates to compositions and methods for the screening, diagnosis and development of therapies for bone-related disorders such as osteoporosis and bone fracture. Antibodies and antibody fragments directed to Ror2 protein are particularly useful in causing dimerization of Ror2 proteins, thereby leading to the activation of Ror2.

### RELATED APPLICATIONS

The present application claims priority under 35 U.S.C. § 119(e) to U.S. provisional patent applications, U.S. Ser. No. 60/774,534, filed Feb. 17, 2006, and U.S. Ser. No. 60/844,239, filed Sep. 13, 2006, which is incorporated herein by reference.

### BACKGROUND OF THE INVENTION

The topic of bone-related disorders and diseases has gained considerable attention over the past years. Bone-related disorders are characterized by bone loss resulting from an imbalance between bone resorption and bone formation. Throughout life, there is a constant remodeling of skeletal bone. In this remodeling process, there is a delicate balance between bone resorption by osteoclasts and bone formation by osteoblasts. Osteoblasts, involved in both endochondral and intramembraneous ossification, are the specialized cells in bone tissue that make matrix proteins that result in the formation of new bone. Bone formation, i.e., osteogenesis, is essential for the maintenance of bone mass in the skeleton. Unlike osteoblasts, osteoclasts are associated with bone resorption and removal. In normal bone, the balance between osteoblast-mediated bone formation and osteoclast-mediated bone resorption is maintained through complex regulated interactions.

There are many deficiencies, diseases, and disorders associated with the skeletal system. Examples of a few include, but are not limited to, osteoporosis, bone cancer, arthritis, rickets, bone fracture, periodontal disease, bone segmental defects, osteolytic bone disease, primary and secondary hyperparathyroidism, Paget's disease, osteomalacia, hyperostosis, and osteopetrosis. Identification of the mechanisms involved in osteogenic differentiation and the renewal processes are crucial for the understanding of bone physiology and skeletal disorders, such as osteoporosis. These disorders may involve deficient bone formation due to defective maturation of putative osteoblastic progenitors.

There exists a need to develop methods of treating diseases or disorders associated with bone resorption and formation, methods of promoting bone formation, methods of identifying agents that modulate (increase or decrease) bone formation, methods of identifying agents that modulate (increase or decrease) bone resorption, and methods of identifying genes or their protein products associated with bone related disorders.

Identification of the mechanisms involved in bone formation and bone resorption are crucial for the understanding of bone physiology and skeletal disorders, such as osteoporosis. The genes or their protein products which are associated with bone related disorders may be used for the elucidation of the molecular mechanisms of bone formation, bone resorption, for the screening and development of new drugs, for diagnosis, prognosis, prevention, and treatment of bone development and bone loss disorders, and evaluation of therapies for bone-related disorders such as osteoporosis. The identified genes and proteins may also be useful in the search for pharmaceutical agents that modulate bone formation. One such protein which has recently been identified is Ror2 protein. Down-regulation of Ror2 gene expression inhibits dexamethasone-induced osteogenic differentiation of human mesenchymal stem cells (FIG. 1) whereas Ror2 over-expression promotes osteogenic differentiation of these cells (Billiard et al., U.S. patent application U.S. Ser. No. 10/823,998, filed Apr. 14, 2004; incorporated herein by reference). Therefore, Ror2 and the Ror2 pathway are suitable targets for modulating bone formation.

### SUMMARY OF THE INVENTION

The present invention provides a system for modulating bone formation. The system is based on the discovery of the role of Ror2 in osteoblast differentiation. In particular, the activation of Ror2 protein leads to mineralized bone formation. Ror2 expression has been found to be crucial in the osteogenic differentiation of mesenchymal stem cells. Ror2 overexpression has also been found to inhibit the differentiation of mesenchymal stem cells into adipocytes. It has also been found that activation of Ror2 protein leads to the phosphorylation of 14-3-3β. The down regulation of 14-3-3β has been found to increase mineralized matrix formation in human mesenchymal stem cells. These discoveries regarding Ror2 protein, its interaction with 14-3-3β protein, and the down-regulation of 14-3-3β make Ror2, 14-3-3β, and other downstream signaling biomolecules prime targets in the search for novel agents that modulate bone formation. Agents that activate Ror2 protein, inhibit 14-3-3β protein, or modulate the activity of other targets downstream are useful in the treatment and prevention of bone-related disorders, particularly disorders associated with bone loss. These agent are also useful in promoting osteoblast differentiation and in promoting mineralized matrix formation. Alternatively, these agents may also find use in inhibiting the differentiation of stem cells into adipocytes and may be useful in treating obesity, metabolic disorders, or diabetes.

The present invention provides agents that activate Ror2 protein. In certain embodiments, the agents cause the dimerization of Ror2 protein thereby leading to Ror2 activation. The dimerization of Ror2 protein leads to increased kinase activity and the subsequent phosphorylation of Ror2's binding partners including 14-3-3β protein. The agent also leads to the promotion of bone growth.

In another aspect, the invention provides agents that inhibit or down-regulate 14-3-3 activity, specifically 14-3-3β or 14-3-3γ. These agents may act at the DNA or protein level to reduce the activity of 14-3-3 in the cells. In certain embodiments, the agent is targeted to cells involved in bone formation such as osteoblasts, mesenchymal stem cells, embryonic stem cells, fetal stem cells, osteo-progenitor cells, pre-osteoblasts, mature osteoblasts, or any other cells in the osteoblast lineage. In other embodiments, the agents is targeted to cells involved in adipocyte formation. For example, the agent may be conjugated to a bisphosphonate moiety to target bone. The down-regulation of 14-3-3β has been found to increase mineralized matrix formation. The agents that target 14-3-3 or specific isoforms of 14-3-3 may be used in conjunction with agents that activate Ror2 protein (e.g., agents that cause the dimerization of Ror2 protein). Such a combination may have synergistic effects in promoting bone formation.

In yet other aspects, the invention provides agents that regulate other downstream elements of the Ror2/14-3-3β pathway that has been found to be important in regulating bone formation. These agents may be used alone or in combination with other agents described herein.

The inventive agents may be any type of chemical compound although proteins, peptides, polynucleotides, and small molecules are preferred. In certain embodiments, the agent is an antibody or fragment thereof which promotes the dimerization of Ror2 protein. The antibody may be polyclonal or monoclonal; however, humanized monoclonal antibodies are typically preferred for the treatment of human subjects. The antibody or fragment thereof may be of any isotype; however, the IgG isotype is generally preferred. In certain embodiments, the agent is a bivalent or multivalent antibody fragment directed to Ror2 protein. In other embodiments, the agent is an 14-3-3β-specific RNAi, siRNA, or shRNA construct.

In one aspect, an agent that activates Ror2 protein or inhibits 14-3-3β activity is administered to a subject to promote bone formation. In certain embodiments, a combination of an agent that activates Ror2 protein and an agent that inhibits 14-3-3β activity is administered to a subject. Specifically, administration of the agent promotes osteoblast differentiation and/or increases mineralized matrix formation. The subject may suffer from or be at risk for any bone-related disorder including osteoporosis, bone cancer, arthritis, rickets, bone fracture, periodontal disease, bone segmental defects, osteolytic bone disease, primary and secondary hyperparathyroidism, Paget's disease, osteomalacia, and hyperostosis. The agent is particularly useful in treating diseases associated with bone loss. In one aspect, the agent promotes the dimerization of Ror2 protein, thereby activating the Ror2 protein. The agent may be any type of chemical compound; however, small molecules, polynucleotides, proteins, and peptides are particularly useful. In certain embodiments, the agent is an antibody or antibody fragment directed to Ror2 protein. Humanized monoclonal are generally preferred given the successful use of humanized monoclonal antibodies in the treatment of human diseases such as, for example, Crohn's disease and multiple sclerosis. In certain embodiments, the agent down-regulates 14-3-3 expression, specifically 14-3-3β or 14-3-3γ expression. In certain particular embodiments, the agent is a 14-3-3β-specific RNAi, shRNA, or siRNA. The inventive agents may also be used o treat obesity, metabolic disorders, or diabetes by promoting osteoblast differentiation at the expense of adipocyte differentiation.

In another aspect, a cell is contacted with an agent that activates Ror2 protein or inhibits 14-3-3β activity to promote osteoblast differentiation or osteogenic differentiation. The cell being contacted typically expresses Ror2 protein or 14-3-3β protein and is capable of undergoing differentiation to the osteoblast phenotype. In certain embodiments, the cell is a stem cell, for example, a mesenchymal stem cell. The cell may be contacted with the agent in vivo or in vitro. The cell may also be contacted with the agent ex vivo and then introduced into a subject in need thereof (e.g., a subject suffering from a bone-related disorder, particularly one associated with bone loss).

Ror2 overexpression and 14-3-3β inhibition have also been found to inhibit adipogenic differentiation. Therefore, agents that activate Ror2 protein or inhibit 14-3-3β activity are also useful in inhibiting adipogenic differentiation. The inventive agents are therefore particularly useful in inhibiting the adipogenic differentiation of stem cells, e.g., mesenchymal stem cells. Based on this discovery, Ror2 activators or 14-3-3β down-regulators may be useful in the treatment or prevention of obesity, diabetes, or other metabolic disorders.

The present invention also includes a system for identifying agents that modulate Ror2 activity or expression or modulate the phosphorylation of 14-3-3β protein. The screening system includes contacting Ror2 protein with an agent and detecting an effect of the agent on Ror2 activity or expression. Detection of an increase in Ror2 activity or expression is indicative of an agent being useful in promoting bone formation, promoting osteoblast differentiation, or inhibiting adipogenic differentiation. In certain embodiments, the activity of Ror2 protein is assayed by determining the extent of dimerization of Ror2 protein. In other embodiments, Ror2 activity is assessed by determining the phosphorylation status of Ror2 protein itself or 14-3-3β. In other embodiments, the Ror2 kinase activity is measured, for example, using ³²P γ ATP or immunoprecipitation using an anti-phosphotyrosine antibody. In certain embodiments, the assay is a cell based assay using a cell expressing Ror2 protein. In other embodiments, the assay is cell-free, and purified or semi-purified Ror2 protein is used. Agents identified using the inventive screening methods and pharmaceutical compositions thereof are particularly useful in the inventive treatment methods described herein.

The present invention also provides an assay for identifying agents that promote the dimerization of Ror2 protein. The assay is particularly amenable to high throughput techniques for screening large numbers of prospective compounds. A chimeric receptor consisting of the extracellular domain of the Ror2 protein is fused to the intracellular domain of TrkB. Agents that dimerize the extracellular Ror2 domains activate the TrkB signaling pathway resulting in an increase in CRE promoter activity. A reporter gene such as luciferase operably linked to the CRE promoter can then be used to identify compounds that dimerize Ror2. The Ror2-TrkB chimera assay has been validated using anti-Ror2 antibodies that have been previously shown to dimerize Ror2. Ror2-specific antibodies cause a dose-dependent increase in observed luciferase activity when compared to cells treated with non-specific IgG (see FIG. 12). The assay provides a rapid, high throughput and highly sensitive assay for identifying agents that activate Ror2. As would be appreciated by those of skill in this art, other intracellular domains besides TrkB may be used to prepare the chimera. A different promoter operably linked to the reporter gene may then be needed in the assay. Agents identified as activators or dimerizers of Ror2 by the inventive assay are also considered part of the present invention.

### DEFINITIONS

The following definitions are provided for a full understanding of terms and abbreviations used herein.

As used herein and in the appended claims, the singular forms “a”, “an”, and “the” include the plural reference unless the context clearly indicates otherwise. Thus, for example, a reference to “a cell” includes a plurality of such cells, and a reference to “an antibody” is a reference to one or more antibodies and equivalents thereof known to those skilled in the art, and so forth.

The abbreviations in the specification correspond to units of measure, techniques, properties or compounds as follows: “g” means gram(s), “mg” means milligram(s), “ng” means nanogram(s), “kDa” means kilodalton(s), “°C” means degree(s) Celsius, “cm” means centimeter(s), “s” means second(s), “min” means minute(s), “h” means hour(s), “l” means liter(s), “ml” means milliliter(s), “μl” means microliter(s), “pl” means picoliter(s), “M” means molar, “mM” means millimolar, “mmole” means millimole(s), “kb” means kilobase(s), “bp” means base pair(s), and “RT” means room temperature.

“High performance liquid chromatography” is abbreviated HPLC.

“Open reading frame” is abbreviated ORF.

“Mass-spectroscopy” is abbreviated MS.

“Tandem mass-spectroscopy” is abbreviated MS/MS.

“Polyacrylamide gel electrophoresis” is abbreviated PAGE.

“Polymerase chain reaction” is abbreviated PCR.

“Reverse transcriptase polymerase chain reaction” is abbreviated RT-PCR.

“Sodium dodecyl sulfate” is abbreviated SDS.

“Sodium dodecyl sulfate-polyacrylamide gel electrophoresis” is abbreviated SDS-PAGE.

“Adenine nucleotide translocator 2” is abbreviated ADP/ATP carrier protein.

“Bone Mineral Density” is abbreviated BMD.

“Ribosomal RNA” is abbreviated rRNA”.

“Untranslated region” is abbreviated UTR.

In the context of this disclosure, a number of terms shall be utilized. As used herein, the term Ror refers to a family of receptor tyrosine kinase-like orphan receptors.

“Ror molecule” refers to Ror polypeptides, Ror proteins, Ror peptides, fragments, variants, and mutants thereof as well as to nucleic acids that encode Ror polypeptides, Ror proteins, Ror peptides and fragments or variants or mutants thereof. “Ror molecule” also refers to Ror polynucleotides, genes and variants and mutants thereof. “Ror molecule” and “Ror” refer to both Ror1 and Ror2 molecules.

“Target Ror molecule” refers to an Ror molecule whose activity is modulated by an agent of the present invention. The target Ror molecule can be Ror polypeptide, homologues, derivatives or fragments or variants or mutants thereof. Ror molecule of interest can also be nucleic acid (oligonucleotide or polynucleotide of RNA or DNA). For example, if proteins of the Ror genes are of interest in an experiment, the target Ror molecules would be the proteins. It is to be understood that the term target Ror molecule refers to both full-length molecules and to fragments, variants, and mutants thereof, such as an epitope of a protein. The target Ror molecule may be either Ror1 molecule or Ror2 molecule or both. In certain particular embodiments, the target Ror molecule is Ror2 protein.

The term “14-3-3” refers to a family of proteins which are involved in signal transduction. The 14-3-3 proteins have a large number of binding partners and are involved in a large and diverse group of cellular processes. The 14-3-3 proteins exert their effects by binding to their target and causing (1) conformational changes; (2) physical occlusion of sequence-specific or structural protein features; and/or (3) scaffolding. Several review articles on the structure and function of 14-3-3 proteins are Bridges and Moorhead, “14-3-3 Proteins: A Number of Functions for a Numbered Protein,” Sci. STKE re10, 2004; Mackintosh, “Dynamic interactions between 14-3-3 proteins and phosphoproteins regulate diverse cellular processes,” Biochem. J. 381:329-42, 2004; each of which is incorporated herein by reference. “14-3-3” may refer to 14-3-3 polypeptides, 14-3-3 proteins, 14-3-3 peptides, 14-3-3 fragments, 14-3-3 variants, and 14-3-3 mutants thereof as well as to nucleic acids that encode 14-3-3 polypeptides, 14-3-3 proteins, 14-3-3 peptides and 14-3-3 fragments or 14-3-3 variants or 14-3-3 mutants thereof. Several isoforms of 14-3-3 have been identified including β, ε, η, γ, τ, ζ, and σ. The activation of Ror2 protein has been found to lead to the phosphorylation of the isoform 14-3-3β. Both 14-3-3β and 14-3-3γ have been found to interact with Ror2. In certain instances, 14-3-3β is referred to specifically. In certain other instances, 14-3-3γ is referred to specifically.

The term “nucleic acid molecule” refers to the phosphate ester form of ribonucleotides (RNA molecules) or deoxyribonucleotides (DNA molecules), or any phosphodiester analogs, in either single-stranded form, or a double-stranded helix. Double-stranded DNA-DNA, DNA-RNA and RNA-RNA helices are possible. The term nucleic acid molecule, and in particular DNA or RNA molecule, refers to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described according to the normal convention of giving only the sequence in the 5′ to 3′ direction along the nontranscribed strand of DNA (i.e., the strand having a sequence homologous to the mRNA).

A “recombinant nucleic acid molecule” is a nucleic acid molecule that has undergone a molecular biological manipulation, i.e., non-naturally occurring nucleic acid molecule or genetically engineered nucleic acid molecule. Furthermore, the term “recombinant DNA molecule” refers to a nucleic acid sequence which is not naturally occurring, or can be made by the artificial combination of two otherwise separated segments of nucleic acid sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, open reading frames, or other useful features may be incorporated by design. Examples of recombinant nucleic acid molecule include recombinant vectors, such as cloning or expression vectors which contain DNA sequences encoding Ror family proteins or immunoglobulin proteins which are in a 5′ to 3′ (sense) orientation or in a 3′ to 5′ (antisense) orientation.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example, thio-uracil, thio-guanine, and fluoro-uracil, or containing carbohydrate, or lipids.

Polynucleotides of the invention may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988), etc. A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, Nature, 290, 304-310, (1981), the promoter contained in the 3′ long terminal repeat of Rous sarcoma virus, Yamamoto et al., Cell, 22, 787-797, (1980), the herpes thymidine kinase promoter, Wagner et al., Proc. Natl. Acad. Sci. U.S.A. 78, 1441-1445, (1981), the regulatory sequences of the metallothionein gene Brinster et al., Nature 296, 39-42, (1982), etc. Any type of plasmid, cosmid, yeast artificial chromosome or viral vector can be used to prepare the recombinant DNA construct that can be introduced directly into the tissue site. Alternatively, viral vectors can be used which selectively infect the desired tissue, in which case administration may be accomplished by another route (e.g., systemically).

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

“RNA transcript” refers to the product resulting from RNA polymerase-catalyzed transcription of a DNA sequence. When the RNA transcript is a complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “polynucleotide encoding polypeptide” encompasses a polynucleotide that may include only the coding sequence as well as a polynucleotide that may include additional coding or non-coding sequence.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength, Sambrook, J. et al. eds., Molecular Cloning: A Laboratory Manual (2d Ed. 1989) Cold Spring Harbor Laboratory Press, NY. Vols. 1-3 (ISBN 0-87969-309-6). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived, Sambrook et al. eds., Molecular Cloning. A Laboratory Manual (2d Ed. 1989) Cold Spring Harbor Laboratory Press, NY. Vols. 1-3. (ISBN 0-87969-309-6), 9.50-9.51). For hybridization with shorter nucleic acids, i.e., oligonucleotides, the position of mismatches becomes more important, and the length of the oligonucleotide determines its specificity, Sambrook et al. eds., Molecular Cloning: A Laboratory Manual (2d Ed. 1989) Cold Spring Harbor Laboratory Press, NY. Vols. 1-3. (ISBN 0-87969-309-6, 11.7-11.8).

The term “complementary” is used to describe the relationship between nucleotide bases that are capable to hybridizing to one another. For example, with respect to DNA, adenosine is complementary to thymine and cytosine is complementary to guanine.

“Identity” or “similarity”, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. Methods commonly employed to determine identity or similarity between sequences include, but are not limited to, those disclosed in Carillo, H., and Lipman, D., SIAM J Applied Math., 48:1073 (1988). Methods to determine identity and similarity are codified in publicly available computer programs. Preferred computer program methods to determine identity and similarity between two sequences include, but are not limited to, GCG program package, Devereux, J., et al., Nucleic Acids Research, 12(1), 387 (1984)), BLASTP, BLASTN, and FASTA Atschul, S. F. et al., J Molec. Biol., 215, 403 (1990)).

“Homologous” refers to the degree of sequence similarity between two polymers (i.e. polypeptide molecules or nucleic acid molecules). The homology percentage figures referred to herein reflect the maximal homology possible between the two polymers, i.e., the percent homology when the two polymers are so aligned as to have the greatest number of matched (homologous) positions.

The term “percent homology” refers to the extent of amino acid sequence identity between polypeptides. The homology between any two polypeptides is a direct function of the total number of matching amino acids at a given position in either sequence, e.g., if half of the total number of amino acids in either of the sequences are the same then the two sequences are said to exhibit 50% homology.

The term “fragment”, “analog”, and “derivative” when referring to polypeptides refers to a polypeptide which may retain essentially the same biological function or activity as the original polypeptide. Thus, an analog includes a precursor protein that can be activated by cleavage of the precursor protein portion to produce an active mature polypeptide. The fragment, analog, or derivative of the polypeptide may be one in which one or more of the amino acids are substituted with a conserved or non-conserved amino acid residues and such amino acid residues may or may not be the ones encoded by the genetic code, or the ones in which one or more of the amino acid residues include a substituent group, or the ones in which the polypeptide is fused with a compound such as polyethylene glycol to increase the half-life of the polypeptide, or the ones in which additional amino acids are fused to the polypeptide such as a signal peptide or a sequence such as polyhistidine tag which is employed for the purification of the polypeptide or the precursor protein. Such fragments, analogs, or derivatives are deemed to be within the scope of the present invention.

“Conserved” residues of a polynucleotide sequence are those residues that occur unaltered in the same position of two or more related sequences being compared. Residues that are relatively conserved are those that are conserved amongst more related sequences than residues appearing elsewhere in the sequences.

Related polynucleotides are polynucleotides that share a significant proportion of identical residues.

Different polynucleotides “correspond” to each other if one is ultimately derived from another. For example, messenger RNA corresponds to the gene from which it is transcribed. cDNA corresponds to the RNA from which it has been produced, such as by a reverse transcription reaction, or by chemical synthesis of a DNA based upon knowledge of the RNA sequence. cDNA also corresponds to the gene that encodes the RNA. Polynucleotides also “correspond” to each other if they serve a similar function, such as encoding a related polypeptide in different species, strains or variants that are being compared.

An “analog” of a DNA, RNA or a polynucleotide, refers to a molecule resembling naturally occurring polynucleotides in form and/or function (e.g. in the ability to engage in sequence-specific hydrogen bonding to base pairs on a complementary polynucleotide sequence) but which differs from DNA or RNA in, for example, the possession of an unusual or non-natural base or an altered backbone. See for example, Uhlmann et al., Chemical Reviews 90, 543-584, (1990).

A “coding sequence” or a sequence “encoding” an expression product, such as an RNA, polypeptide, or protein, is a nucleotide sequence that, when expressed, results in the production of that RNA, polypeptide, or protein (e.g., enzyme), i.e., the nucleotide sequence encodes an amino acid sequence for that polypeptide or protein.

A “substantial portion” of an amino acid or nucleotide sequence is a portion comprising enough of the amino acid sequence of a polypeptide or the nucleotide sequence of a gene to putatively identify that polypeptide or gene, either by manual evaluation of the sequence by one skilled in the art, or by computer automated sequence comparison and identification using algorithms such as BLAST (Basic Local Aligmnent Search Tool; Altschul, S. F., et al., J. Mol. Biol. 215, 403-410, (1993); see also www.ncbi.nlm.nih.gov/BLAST).

Accordingly, a “substantial portion” of a nucleotide sequence comprises enough of the sequence to specifically identify and/or isolate a nucleic acid fragment comprising the sequence. The skilled artisan, having the benefit of the sequences as reported herein, may now use all or a substantial portion of the disclosed sequences for purposes known to those skilled in this art.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as related to a sequence of DNA, means that the component nucleotides were assembled in vitro. Manual chemical synthesis of DNA may be accomplished using well-known procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determining preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Gene control sequence” refers to the DNA sequences required to initiate gene transcription plus those required to regulate the rate at which initiation occurs. Thus a gene control sequence may consist of the promoter, where the general transcription factors and the polymerase assemble, plus all the regulatory sequences to which gene regulatory proteins bind to control the rate of these assembly processes at the promoter. For example, the control sequences that are suitable for prokaryotes may include a promoter, optionally an operator sequence, and a ribosome-binding site. Eukaryotic cells may utilize promoters, enhancers, and/or polyadenylation signals.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the expression level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions.

The “3′non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The “translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency.

The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (i.e., that the coding sequence is under the transcriptional control of the promoter). Coding sequences can be operatively linked to regulatory sequences in sense or antisense orientation. The term “promoter operable in bone cells” refers to a promoter that is recognized by the RNA polymerase of the bone cell.

“RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from posttranscriptional processing of the primary transcript and is referred to as the mature RNA. “Messenger RNA (mRNA)” refers to the RNA that is without introns and that can be translated into polypeptide. “cDNA” refers to a double-stranded DNA that is complementary to and derived from mRNA. “Sense” RNA refers to an RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “expression” refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein.

“Overexpression” refers to the production of a gene product in an organism that exceeds levels of production in normal or non-transformed organisms. “Suppression” refers to suppressing the expression of foreign or endogenous genes or RNA transcripts.

“Altered levels” refers to the production of gene product(s) in organisms in amounts or proportions that differ from that of normal or non-transformed organisms. Overexpression of the polypeptide of the present invention may be accomplished by first constructing a chimeric gene or chimeric construct in which the coding region is operatively linked to a promoter capable of directing expression of a gene or construct in the desired tissues at the desired stage of development. For reasons of convenience, the chimeric gene or chimeric construct may comprise promoter sequences and translation leader sequences derived from the same genes. 3′ Non-coding sequences encoding transcription termination signals may also be provided. The instant chimeric gene or chimeric construct may also comprise one or more introns in order to facilitate gene expression. Plasmid vectors comprising the instant chimeric gene or chimeric construct can then be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene or chimeric construct. The skilled artisan will also recognize that different independent transformation events will result in different levels and patterns of expression, Jones et al., EMBO J., 4, 2411-2418, (1985); De Almeida et al., Mol. Gen. Genetics, 218, 78-86, (1989), and thus that multiple events must be screened in order to obtain lines displaying the desired expression level and pattern. Such screening may be accomplished by southern analysis of DNA, northern analysis of mRNA expression, western or immunocytochemical analysis of protein expression, or phenotypic analysis.

The terms “variant” or “variants” refer to variations of the nucleic acid or amino acid sequences of Ror molecule. Encompassed within the term “variant(s)” are nucleotide and amino acid substitutions, additions, or deletions of Ror molecules. Also, encompassed within the term “variant(s)” are chemically modified natural and synthetic Ror molecules. For example, variant may refer to polypeptides that differ from a reference polypeptide. Generally, the differences between the polypeptide that differs in amino acid sequence from reference polypeptide, and the reference polypeptide are limited so that the amino acid sequences of the reference and the variant are closely similar overall and, in some regions, identical. A variant and reference polypeptide may differ in amino acid sequence by one or more substitutions, deletions, additions, fusions and truncations that may be conservative or non-conservative and may be present in any combination. For example, variants may be those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination. Additionally, a variant may be a fragment of a polypeptide of the invention that differs from a reference polypeptide sequence by being shorter than the reference sequence, such as by a terminal or internal deletion. A variant of a polypeptide of the invention also includes a polypeptide which retains essentially the same biological function or activity as such polypeptide, e.g., precursor proteins which can be activated by cleavage of the precursor portion to produce an active mature polypeptide. These variants may be allelic variations characterized by differences in the nucleotide sequences of the structural gene coding for the protein, or may involve differential splicing or post-translational modification. Variants also include a related protein having substantially the same biological activity, but obtained from a different species. The skilled artisan can produce variants having single or multiple amino acid substitutions, deletions, additions, or replacements. These variants may include, inter alia: (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more amino acids are deleted from the peptide or protein, or (iii) one in which one or more amino acids are added to the polypeptide or protein, or (iv) one in which one or more of the amino acid residues include a substituent group, or (v) one in which the mature polypeptide is fused with another compound, such as a compound to increase the half-life of the polypeptide (for example, polyethylene glycol), or (vi) one in which the additional amino acids are fused to the mature polypeptide such as a leader or secretory sequence or a sequence which is employed for purification of the mature polypeptide or a precursor protein sequence. A variant of the polypeptide may also be a naturally occurring variant such as a naturally occurring allelic variant, or it may be a variant that is not known to occur naturally. All such variants defined above are deemed to be within the scope of teachings in the art.

The polypeptides and the polynucleotides of the present invention are preferably provided in an isolated form, and may be purified to homogeneity. The polypeptides and polynucleotides in certain instances are at least 90% pure, at least 95% pure, at least 98% pure, or at least 99% pure.

The term “isolated” means that the material is removed from its original or native environment (e.g., the natural environment if it is naturally occurring). Therefore, a naturally-occurring polynucleotide or polypeptide present in a living animal is not isolated, but the same polynucleotide or polypeptide, separated by human intervention from some or all of the coexisting materials in the natural system, is isolated. For example, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA and combined with carbohydrate, lipid, protein or other materials. Such polynucleotides could be part of a vector and/or such polynucleotides or polypeptides could be part of a composition, and still be isolated in that such vector or composition is not part of the environment in which it is found in nature. Similarly, the term “substantially purified” refers to a substance, which has been separated or otherwise removed, through human intervention, from the immediate chemical environment in which it occurs in Nature. Substantially purified polypeptides or nucleic acids may be obtained or produced by any of a number of techniques and procedures generally known in the field.

The term “purification” refers to increasing the specific activity or concentration of a particular polypeptide or polypeptides in a sample. In one embodiment, specific activity is expressed as the ratio between the activity of the target polypeptide and the concentration of total polypeptide in the sample. In another embodiment, specific activity is expressed as the ratio between the concentration of the target polypeptide and the concentration of total polypeptide. Purification methods include but are not limited to dialysis, centrifugation, and column chromatography techniques, which are well-known procedures to those of skill in the art. See, e.g., Young et al., 1997, “Production of biopharmaceutical proteins in the milk of transgenic dairy animals,” BioPharm 10(6): 34-38.

The terms “substantially pure” and “isolated” are not intended to exclude mixtures of polynucleotides or polypeptides with substances that are not associated with the polynucleotides or polypeptides in nature.

The terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell (e.g., bacterial cells such as E. coli, yeast cells, mammalian cells, avian cells, amphibian cells, plant cells, fish cells, and insect cells), whether located in vitro or in vivo. For example, host and may include any transformable organisms that are capable of replicating a cells may be located in a transgenic animal. Host cell can be used as a recipient for vectors vector and/or expressing a heterologous nucleic acid encoded by a vector.

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Transformation of insect cells and production of foreign polypeptides therein is disclosed by Guarino et al., U.S. Pat. No. 5,162,222 and WIPO publication WO94/06463. Methods for isolating recombinant proteins from a baculovirus system are also described by Richardson (ed.), “Baculovirus Expression Protocols” (The Humana Press, Inc. 1995). In one embodiment, the polypeptides of the invention can be expressed using a baculovirus expression system (see, Luckow et al., Bio/Technology, 1988, 6, 47, “Baculovirus Expression Vectors: a Laboratory Manual”, O'Rielly et al. (Eds.), W. H. Freeman and Company, New York, 1992, U.S. Pat. No. 4,879,236, each of which is incorporated herein by reference in its entirety). In addition, the MAXBAC™ complete baculovirus expression system (Invitrogen) can, for example, be used for production in insect cells.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182:529 (1990)). Within additional embodiments of the invention, a fusion of the polypeptide of interest and an affinity tag (e.g., maltose-binding protein, an immunoglobulin domain) may be constructed to facilitate purification.

Host cells of the invention can be used in methods for the large-scale production of Ror polypeptides wherein the cells are grown in a suitable culture medium and the desired polypeptide products are isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

The term “in vitro” refers to an artificial environment and to reactions or processes that occur within an artificial environment. In vitro environments include, but are not limited to, test tubes and cell cultures. The term “in vivo” refers to the natural environment (e.g., an animal or a cell) and to processes or reaction that occur within a natural environment.

The methods of the present invention may be performed in vitro using cells (cultured cells) and cell lysates, including nuclear extracts. Examples of cells contemplated for identifying agents that modulate bone formation include, but are not limited to, calvarial cells, osteoblasts, osteoclasts, chondrocytes, and pluripotent precursor cells, such as multipotent bone marrow stromal cells. Specific examples of osteoblast and osteoblast precursor cell lines include MC3T3-E1, C2C12, MG-63 cells, U2OS cells, UMR106 cells, ROS17/2.8 cells, SaOS-2 cells, and the like that are provided in the catalog from the ATCC (WO 01/19855) as well as HOB cell lines described in Bodine P V, Vernon S K, Komm B S., Endocrinology, 137, 4592-4604, (1996), Bodine P V N, TrailSmith M, Komm B S., J Bone Min Res, 11, 806-819, (1996), Bodine P V, Green J, Harris H A, Bhat R A, Stein G S, Lian J B, Komm B S., J Cell Biochem, 65, 368-387, (1997), Bodine P V, Komm B S., Bone, 25, 535-43 (1999), Bodine P V N, Harris H A, Komm B S., Endocrinology, 140, 2439-2451, (1999), Prince M, Banerjee C, Javed A, Green J, Lian J B, Stein G S, Bodine P V, Komm B S, J Cell Biochem, 80, 424-40, (2001). The methods of the present invention may also be performed using a cell-free system.

The term “expression system” refers to a host cell and compatible vector under suitable conditions, e.g., for the expression of a protein coded for by foreign DNA carried by the vector and introduced into the host cell. Common expression systems include E. coli host cells and plasmid vectors, insect host cells and Baculovirus vectors, and mammalian host cells and vectors.

“Transformation” refers to the transfer of a nucleic acid fragment into the genome of a host organism, resulting in genetically stable inheritance. Host organisms containing the transformed nucleic acid fragments are referred to as “transgenic” organisms.

The term “differentiate” refers to having a different character or function from the original type of tissues or cells. Thus, “differentiation” is the process or act of differentiating.

The term “osteoblast differentiation” refers to the process in which a cell develops specialized functions during maturation into an osteoblast cell. Osteoblast differentiation may include pre-osteoblast, early and mature osteoblast, pre-osteocyte and mature osteocyte stages (Bodine et al, Vitamins and Hormones 65, 101-151 (2002), Stein et al. Endocrine Reviews 14, 424-442 (1993), and Lian et al. Vitamins and Hormones 55, 443-509 (1999)).

The term “proliferation” refers to the growth and production of similar cells.

The term “phenotype” refers to the observable character of a cell or an organism. Such observable character can involve the physical appearance, as well as a level of particular physiological compositions present in the cell or organism. Ostoeblastic phenotype includes expression of several marker proteins such as bone-specific transcription factor Cbfal; type I collagen; alkaline phosphatase, osteocalcin; and bone sialoprotein.

As used herein, the term “binding partner” or “interacting proteins” refer to a molecule capable of binding another molecule with specificity, as for example, an antigen and an antigen-specific antibody or an enzyme and its inhibitor. Binding partners may include, for example, biotin and avidin or streptavidin, IgG and protein A, receptor-ligand couples, protein-protein interaction, and complementary polynucleotide strands. The term “binding partner” may also refer to polypeptides, lipids, small molecules, or nucleic acids that bind to kinases in cells. A change in the interaction between a kinase and a binding partner can manifest itself as an increased or decreased probability that the interaction forms, or an increased or decreased concentration of kinase-binding partner complex. For example, Ror1 or Ror 2 protein may bind with another protein or polypeptide and form a complex that may result in modulating Ror1 or Ror2 activity.

The term “signal transduction pathway” refers to the molecules that propagate an extracellular signal through the cell membrane to become an intracellular signal. This signal can then stimulate a cellular response. The polypeptide molecules involved in signal transduction processes may be receptor and non-receptor protein tyrosine kinases.

“Receptor” refers to a molecular structure within a cell or on the surface of the cell that is generally characterized by the selective binding of a specific substance. Exemplary receptors include cell-surface receptors for peptide hormones, neurotransmitters, antigens, complement fragments and immunoglobulins as well as cytoplasmic receptors for steroid hormones.

The term “modulate” refers to the suppression, enhancement, or induction of a function. For example, “modulation” or “regulation” of gene expression refers to a change in the activity of a gene. Modulation of expression can include, but is not limited to, gene activation and gene repression. “Modulate” or “regulate” also refers to methods, conditions, or agents which increase or decrease the biological activity of a protein, enzyme, inhibitor, signal transducer, receptor, transcription activator, co-factor, and the like. This change in activity can be an increase or decrease of mRNA translation, DNA transcription, and/or mRNA or protein degradation, which may in turn correspond to an increase or decrease in biological activity. Such enhancement or inhibition may be contingent upon occurrence of a specific event, such as activation of a signal transduction pathway and/or may be manifest only in particular cell types.

“Modulated activity” refers to any activity, condition, disease or phenotype that is modulated by a biologically active form of a protein. Modulation may be affected by affecting the concentration of biologically active protein, e.g., by regulating expression or degradation, or by direct agonistic or antagonistic effect as, for example, through inhibition, activation, binding, or release of substrate, modification either chemically or structurally, or by direct or indirect interaction which may involve additional factors.

“Modulator” refers to any agent that alters the expression of a specific activity, such as bone formation or Ror molecule expression. For example, an agent that modulates bone formation alters or changes (increases or decreases) bone formation. The modulator is intended to comprise any compound, e.g., antibody, small molecule, peptide, oligopeptide, polypeptide, or protein.

“Plasma cell” refers to a mature B lymphocyte that is specialized for antibody (immunoglobulin) production. Plasma cells are rarely found in the peripheral blood. They comprise from 0.2% to 2.8% of the bone marrow white cell count. Mature plasma cells are often oval or fan shaped, measuring 8-15 μm. The nucleus is eccentric and oval in shape.

The term “small molecule” refers to a synthetic or naturally occurring chemical compound, for instance a peptide or oligonucleotide that may optionally be derivatized, natural product or any other low molecular weight (typically less than about 5 kDalton) organic, bioinorganic or inorganic compound, of either natural or synthetic origin. Such small molecules may be a therapeutically deliverable substance or may be further derivatized to facilitate delivery.

As used herein, the term “inducer” refers to any agent that induces, enhances, promotes or increases a specific activity, such as bone formation, or Ror molecule expression.

As used herein the term “inhibitor” or “repressor” refers to any agent that inhibits, suppresses, represses, or decreases a specific activity, such as bone formation, or Ror molecule expression.

As used herein, the term “agent” or “test agent” refers to any compound or molecule that is to be tested. Examples of agents of the present invention include but are not limited to peptides, small molecules, and antibodies. Agents can be randomly selected or rationally selected or designed. As used herein, an agent is said to be “randomly selected” when the agent is chosen randomly without considering the specific interaction between the agent and the target compound or site. As used herein, an agent is said to be “rationally selected or designed”, when the agent is chosen on a non-random basis which takes into account the specific interaction between the agent and the target compound or site and/or the conformation in connection with the agent's action.

As used herein, the term “antibody” refers to an immunoglobulin molecule or an immunologically active portion thereof (e.g., antigen-binding portion). The antibody be naturally produced or wholly or partially synthetically produced. Examples of immunologically active portion of immunoglobulin molecules include F(ab), Fv, and F(ab′) fragments which can be generated by cleaving the antibody with an enzyme such as pepsin. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are generally preferred in the present invention.

The term “antibody fragment” refers to any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody, or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages or other more stable linkages. The fragment may also optionally be a multimolecular complex. A functional antibody fragment will typically comprise at least about 50 amino acids and more typically will comprise at least about 200 amino acids. In certain embodiments, the antibody fragment has at least two antigen-binding site. In certain preferred embodiments, the antibody fragment has exactly 2, 3, 4, or 5 antigen-binding sites. Fragments with two antigen-binding sites are particularly useful in the present invention. Such agents dimerize Ror2 without the formation of multimeric complexes.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

Diabodies are dimeric scFvs. The components of diabodies typically have shorter peptide linkers than most scFvs, and they show a preference for associating as dimers.

An Fv fragment is an antibody fragment which consists of one V_{H} and one V_{L} domain held together by noncovalent interactions. The term dsFv is used herein to refer to an Fv with an engineered intermolecular disulfide bond to stabilize the V_{H}-V_{L} pair.

A F(ab′)₂ fragment is an antibody fragment essentially equivalent to that obtained from immunoglobulins (typically IgG) by digestion with an enzyme pepsin at pH 4.0-4.5. The fragment may be recombinantly produced.

A Fab fragment is an antibody fragment essentially equivalent to that obtained by reduction of the disulfide bridge or bridges joining the two heavy chain pieces in the F(ab′)₂ fragment. The Fab′ fragment may be recombinantly produced.

A Fab fragment is an antibody fragment essentially equivalent to that obtained by digestion of immunoglobulins (typically IgG) with the enzyme papain. The Fab fragment may be recombinantly produced. The heavy chain segment of the Fab fragment is the Fd piece.

The term “reporter gene,” as used herein, refers to any gene whose phenotypic expression is easy to monitor. The use of reporter genes is particularly useful in screening to determine which test agents activate a signaling pathway. The reporter gene is operably linked to a promoter or other regulatory element that is controlled by the signaling pathway. In certain embodiments, a recombinant DNA construct is made in which the reporter gene is functionally attached to a promoter region or other regulatory region of particular interest, and the construct is transfected into a cell or organism. Examples of commonly used reporter genes include luciferase (LUC), green fluorescent protein (GFP), β-galactosidase (GAL), β-glucuronidase (GUS), and chloramphenicol acetyltransferase (CAT).

As used herein, the terms “treatment”, “treating”, and “therapy” refer to therapeutic treatment and prophylactic, or preventative manipulations, or manipulations which stimulate bone cell differentiation or bone formation, postpone the development of bone disorder symptoms, and/or reduce the severity of bone disorders and/or such symptoms that will or are expected to develop from a bone disorder. The terms further include ameliorating existing bone disorder symptoms, preventing additional symptoms, ameliorating or preventing the underlying metabolic causes of symptoms, preventing or reversing metabolic causes of symptoms, or, preventing or promoting bone growth. Thus, the terms denote that a beneficial result has been conferred on a subject with a bone disorder, or with the potential to develop such disorder. Furthermore, the term “treatment” is defined as the application or administration of an agent (e.g., therapeutic agent or a therapeutic composition) to a subject, or an isolated tissue or cell line from a subject, who may have a disease, a symptom of disease or a predisposition toward a disease, with the purpose to cure, heal, alleviate, relieve, alter, remedy, ameliorate, improve or affect the disease, the symptoms of disease or the predisposition toward disease. As used herein, a “therapeutic agent” refers to any substance or combination of substances that assists in the treatment of a disease, e.g., modulates bone forming activity or induces new bone formation. Accordingly, a therapeutic agent includes, but is not limited to, small molecules, peptides, antibodies, ribozymes and antisense oligonucleotides.

Therapeutic agent or therapeutic compositions may also include a compound in a pharmaceutically acceptable form that prevents and/or reduces the symptoms of a particular disease. For example a therapeutic composition may be a pharmaceutical composition that prevents and/or reduces the symptoms of a bone related disorder. It is contemplated that the therapeutic composition of the present invention will be provided in any suitable form. The form of the therapeutic composition will depend on a number of factors, including the mode of administration. The therapeutic composition may contain diluents, adjuvants and excipients, among other ingredients.

The bone strength may be determined by bone density (grs of mineral/cm³ of volume) and bone quality (mineralization, bone architecture, bone turnover, micro fractures). As a measure for bone strength, Bone Mineral Density (BMD) is usually used. For example, a bone can be declared osteoporotic if its BMD is exceeds 2.5 standard deviations below the mean of BMD of young white adult women (World Health Organization, 1994, Assessment of Fracture Risk and it's Application to Screening for Postmenopausal Osteoporosis. Technical Report Series 843. Geneva: World health Organization).

“Bone tissue” refers to calcified tissues (e.g., calvariae, tibiae, femurs, vertebrae, teeth), bone trabeculae, the bone marrow cavity, which is the cavity other than the bone trabeculae, the cortical bone, which covers the outer peripheries of the bone trabeculae and the bone marrow cavity, and the like. Bone tissue also refers to bone cells that are generally located within a matrix of mineralized collagen; blood vessels that provide nutrition for the bone cells; bone marrow aspirates: joint fluids: bone cells that are derived from bone tissues; and may include fatty bone marrow. Bone tissue includes bone products such as whole bones, sections of whole bone, bone chips, bone powder, bone tissue biopsy, collagen preparations, or mixtures thereof. For the purposes of the present invention, the term “bone tissue” is used to encompass all of the aforementioned bone tissues and products, whether human or animal, unless stated otherwise.

As used herein, “bone-related activity” includes bone-forming activity and bone-resorbing activity. Bone-forming activity can be induced by increasing osteoblastic activity, osteoblastic differentiation from osteoprogenitor cells, and osteoblastic proliferation, by decreasing osteoblast apoptosis and by any combination thereof. In addition, bone-resorbing activity can be suppressed by decreasing osteoclast activity, osteoclast differentiation and proliferation, by increasing osteoclast apoptosis and by any combination thereof. Bone-forming activity can be induced in various bone tissues or cells.

As used herein, the phrase “modulating bone formation” refers to increase or decrease in bone formation. “Increased bone formation” is meant the recruitment of osteoblasts or osteoblast precursors to a bone site, which results in differentiation of the cells inot mature osteoblasts and their secretion of collagenous matrix which mineralizes int bone matter and increases bone mass at the site. The term also encompasses the increased production and secretion of collagenous matrix by mature osteoblasts. Increased bone formation can be determined via one or more of a decrease in fracture rate, an increase in areal bone density, an increase in volumetric mineral bone density, an increase in trabecular connectivity, an increase in trabecular density, an increase in cortical density or thickness, an increase in bone diameter, and an increase in inorganic bone content. Increased bone formation may result from increased attachment, proliferation, survival and/or differentiation of bone cells, e.g., osteoblasts, and subsequent bone mineralization.

“Bone-related disorders” include disorders of bone formation and bone resorption. These diseases and conditions include, but are not limited to, rickets, osteomalacia, osteopenia, osteosclerosis, renal osteodystrophy, osteoporosis (including senile and postmenopausal osteoporosis), Paget's disease, bone metastases, hypercalcaemia, hyperparathyroidism, osteopetrosis, periodontitis, and the abnormal changes in bone metabolism which may accompany rheumatoid arthritis and osteoarthritis. Some of these diseases are characterized by insufficient bone formation or bone loss, while others involve an abnormal thickening or hardening of bone tissue. Examples of diseases that would benefit from inhibiting abnormal thickening of the bone include but are not limited to osteopetrosis and osteosclerosis.

“Bone-related agents” refer to agents that influence bone formation or bone resorption. “Bone-related agents” may induce anabolic or catabolic effect, may inhibit bone resorption and result in increased bone mineral density, may increase bone formation, or may maintain the balance between bone formation and bone resorption.

The terms “compound” or “agent” are used interchangeably herein to refer to a compound or compounds or composition of matter which, when administered to a subject (human or animal) induces a desired pharmacologic and/or physiologic effect by local and/or systemic action.

The term “subject” refers to any mammal, including a human, or non-human subject. Non-human subjects can include experimental, test, agricultural, entertainment or companion animals. A subject may be a human. A subject may be a domesticated animal, such as a dog, cat, cow, goat, sheep, pig, etc., A subject may be an experimental animal, such as a mouse, rat, rabbit, monkey, etc.

The term “biological sample” is broadly defined to include any cell, tissue, biological fluid, organ, multi-cellular organism, and the like. A biological sample may be derived, for example, from cells or tissue cultures in vitro. Alternatively, a biological sample may be derived from a living organism or from a population of single-cell organisms. A biological sample may be a live tissue such as live bone. The term “biological sample” is also intended to include samples such as cells, tissues or biological fluids isolated from a subject, as well as samples present within a subject. That is, the detection method of the invention can be used to detect Ror mRNA, protein, genomic DNA, or activity in a biological sample in vitro as well as in vivo. For example, in vitro techniques for detection of Ror mRNA include TaqMan analysis, northern hybridization, and in situ hybridization. In vitro techniques for detection of Ror protein include enzyme-linked immunosorbent assays (ELISAs), western blots, immunoprecipitations and immunofluorescence. In vitro techniques for detection of Ror genomic DNA include southern hybridizations.

### BRIEF DESCRIPTION OF THE DRAWING

The invention can be more fully understood from the following detailed description and the accompanying figures that form a part of this application.

FIG. 1 shows that down-regulating Ror2 expression inhibits dex-induced osteogenic differentiation of human mesenchymal stem cells (hMSC). Human MSC were infected with adenoviral expression vectors containing Ror2-specific shRNA or EGFP-specific shRNA (control) and incubated in MSC growth medium (MSCGM) supplemented with 0.05 mM ascorbic acid, 10 mM β-glycerophosphate (β-GP) and 100 nM dexamethasone (dex). After 9 days of incubation, the whole cell protein extracts (50 μg/lane) were subjected to western immunoblotting for the endogenous Ror2 protein or β-actin as loading control (A). After 11 days of incubation, alizarin red-S staining was performed (B) and quantified (C) to assess the extent of mineralized matrix formation. In C, the amount of alizarin red-S incorporated in presence of EGFP shRNA was set at 100%. The results in B and C are representative of three independent experiments (FIG. 1 is referred to in Example 1).

FIG. 2 shows that Ror2 over-expression inhibits adipogenic differentiation of hMSC. A. Human MSC were infected with β-galactosidase (β-gal), Ror2, or Ror2KD and incubated in MSCGM supplemented with adipogenic cocktail for 8 days. Total cellular RNA was isolated and subjected to real-time RT-PCR analysis for adipogenic transcription factors C/EBPα and PPARγ using primers and probes obtained from Applied Biosystems. The levels of mRNA were normalized to the expression of cyclophilin B in each sample and the relative mRNA expression in β-gal-infected cells was set at 100%. B. Human MSC were infected with β-gal, Ror2, or Ror2KD, incubated in MSCGM supplemented with adipogenic cocktail for 21 days and then subjected to oil red O staining (FIG. 2 is referred to in Example 2).

FIG. 3 shows that over-expression of Ror2 protein increases total bone area, but not osteoblast number in neonatal mouse calvariae. Calvarial bones of 4 days old mouse littermates were either left uninfected (control) or infected with adenoviruses coding for β-galactosidase (β) or human Ror2 (R2). After 7 days of incubation in presence of adenovirus, calvariae were stained with hematoxylin and eosin prior to assessing total bone area and osteoblast number. Values obtained in uninfected cultures were set at 100%. The results are means ∀ SE of 4-5 calvariae per condition (*−P<0.01 compared to β-galactosidase infection). This graph is representative of 3 independent experiments (FIG. 3 is referred to in Example 3)

FIG. 4 demonstrates that Ror2 protein binds 14-3-3β and phosphorylates it on tyrosine(s). U2OS cells were infected with β-gal (β), Ror2 (R2), or Ror2KD (KD) adenoviruses and 24-48 h later the whole cell lysates were prepared and immunoprecipitated with anti-flag (A), anti-14-3-3β (B), or anti-phosphotyrosine (C) antibodies. The immunoprecipitates were analyzed by immunoblotting with the indicated antibodies (FIG. 4 is referred to in Example 4).

FIG. 5 shows that endogenous Ror2 protein mediates 14-3-3β phosphorylation in vivo. U2OS cells were transiently transfected with Ror2 siRNA or non-specific siRNA and 48 h later total cellular protein extracts were subjected to western immunoblotting for the endogenous Ror2 protein or β-actin (loading control) using 50 μg of extract per lane (A). Same lysates were also analyzed by 14-3-3β antibody directly (20 μg/lane) or after immunoprecipitation with anti-phosphotyrosine antibody (B) (FIG. 5 is referred to in Example 4).

FIG. 6 shows that the cytosolic domain of Ror2 protein binds to and directly phosphorylates 14-3-3β in vitro. A. GST-tagged cytosolic domain of Ror2 (GST-R2c) or GST alone were attached to glutathione sepharose beads and incubated with in vitro translated 14-3-3β for 4 h at 4° C. The bound material was analyzed with anti-14-3-3β antibody. B. In vitro kinase assay performed as described in “General Methods” with purified recombinant cytosolic domain of Ror2 protein (GST-R2c, Invitrogen) and purified recombinant GST-14-3-3β (Biomol, Inc.) (FIG. 6 is referred to in Example 4).

FIG. 7 demonstrates that Ror2-specific antibody causes dimerization and activation of the Ror2 receptor. A. To demonstrate dimerization, U2OS cells were transfected with Ror2 expression plasmids tagged at the COOH terminus with either Flag (R2-F) or His (R2-H) epitope tag. Twenty four hours later, the cells were treated with Ror2-specific goat polyclonal IgG or a non-specific goat IgG for 1 h at 37 C and the whole-cell protein extracts were prepared and immunoprecipitated on anti-Flag affinity agarose. The precipitates were analyzed by immunoblotting with anti-His antibody (top panel). The bottom panel shows the same membrane reprobed with anti-Flag antibody for precipitation level control. B. To demonstrate activation, untransfected U2OS cells were treated with Ror2-specific antibody or control IgG as in A, and the whole-cell extracts were precipitated on anti-phosphotyrosine antibody and analyzed with Ror2 or 14-3-3β antibody (FIG. 7 is referred to in Example 5).

FIG. 8 shows that Ror2 antibody causes mineralized matrix formation in hMSC. Human MSC were incubated in MSCGM containing 0.05 mM ascorbic acid, 10 mM β-GP and 100 nM dex supplemented with either non-specific goat IgG, the Ror1-specific goat IgG (50 μg/ml each), or increasing concentrations of the Ror2-specific goat IgG. The extent of matrix mineralization was assessed after 9 days of incubation by alizarin red-S staining (FIG. 8 is referred to in Example 6).

FIG. 9 demonstrates that the hMSC mineralization induced by Ror2 antibody is mediated through Ror2. A. hMSC were infected with adenoviral expression vectors containing shRNA specific for Ror2 or for EGFP (control) and incubated in MSCGM supplemented with 0.05 mM ascorbic acid, 10 mM β-GP and 100 nM dex. After 9 days of incubation, the extent of matrix mineralization was assessed by alizarin red-S staining. B. Human MSC were infected with Ror2 adenovirus for 24 h and then incubated in MSCGM containing 0.05 mM ascorbic acid, 10 mM β-GP and either Ror2-specific goat IgG or non-specific goat IgG for 19 days prior to staining with alizarin red-S (FIG. 9 is referred to in Example 6).

FIG. 10 demonstrates that down-regulating 14-3-3β enhances mineralized matrix formation in hMSC. Human MSC were infected with adenoviral expression vectors containing scramble shRNA; 14-3-3β-specific shRNA; β-galactosidase (β-gal) over-expression cassette; or Ror2 over-expression cassette and incubated in MSCGM supplemented with 0.05 mM ascorbic acid, 10 mM β-GP and 100 nM dex. After 9 days of incubation, 50 μg of the whole-cell protein extracts were subjected to western immunoblotting for the endogenous 14-3-3β protein (A). After 12 days of incubation, alizarin red-S staining was performed (B) to assess the extent of mineralized matrix formation (FIG. 10 is referred to in Example 7).

FIG. 11 demonstrates that Ror2 antibody treatment and 14-3-3β down-regulation promote new bone formation ex-vivo. Mouse calvarial bones were infected with adenoviruses containing scrambled shRNA (scr) or shRNA specific for 14-3-3β; and 48 h later were treated with 12 μg/ml of anti-Ror2 antibody or non-specific IgG in presence of calcein. After 7 days of incubation with adenoviruses and antibodies, calvariae were stained with hematoxylin-eosin and total bone area (open bars) and osteoblast number (solid bars) were determined. Values obtained in scrambled shRNA-infected and IgG-treated cultures were set at 100%. The results are means ∀ SE of 4 calvariae per condition (*−p<0.05) (FIG. 11 is referred to in Example 8).

FIG. 12 illustrates generation of a high throughput, high sensitivity assay for Ror2 activity. A. Schematic representation of the assay that utilizes the signaling pathway of the TrkB receptor. The TrkB receptor is activated by ligand-induced homo-dimerization that causes phosphorylation of Erk and stimulation of the cAMP response element (CRE) in the promoter of target genes. We generated a chimeric receptor consisting of the extracellular domain of Ror2 (aa 1-407) fused to the transmembrane and intracellular domains of TrkB (aa 432-822). When using this chimera, agents that cause Ror2 dimerization activate TrkB signaling pathway and this activation can be assessed by a CRE promoter-luciferase reporter assay. B. HEK293 cells were stably transfected with the Ror2-TrkB chimera and CRE-luciferase plasmids, treated with the indicated amounts of the anti-Ror2 antibody or non-specific IgG for 24 h, and luciferase activity was assessed. Luciferase activity observed upon treatment with the non-specific IgG was set at 1. The results are representative of three independent experiments (means ∀ SE; n=4; *−p<0.05) (FIG. 12 is referred to in Example 9).

### DETAILED DESCRIPTION OF CERTAIN PREFERRED EMBODIMENTS OF THE INVENTION

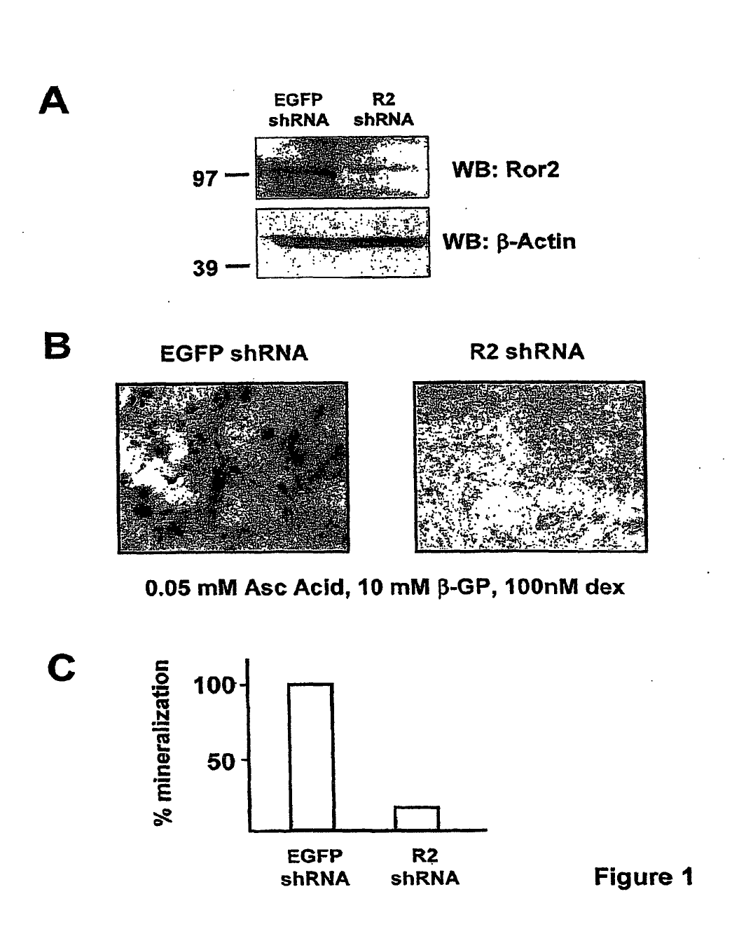

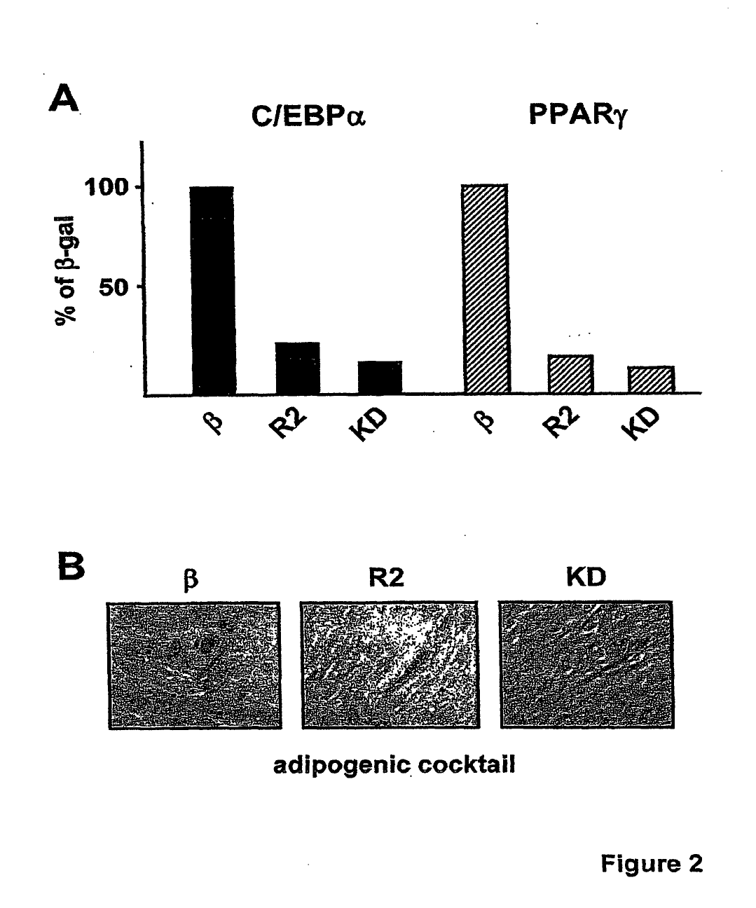

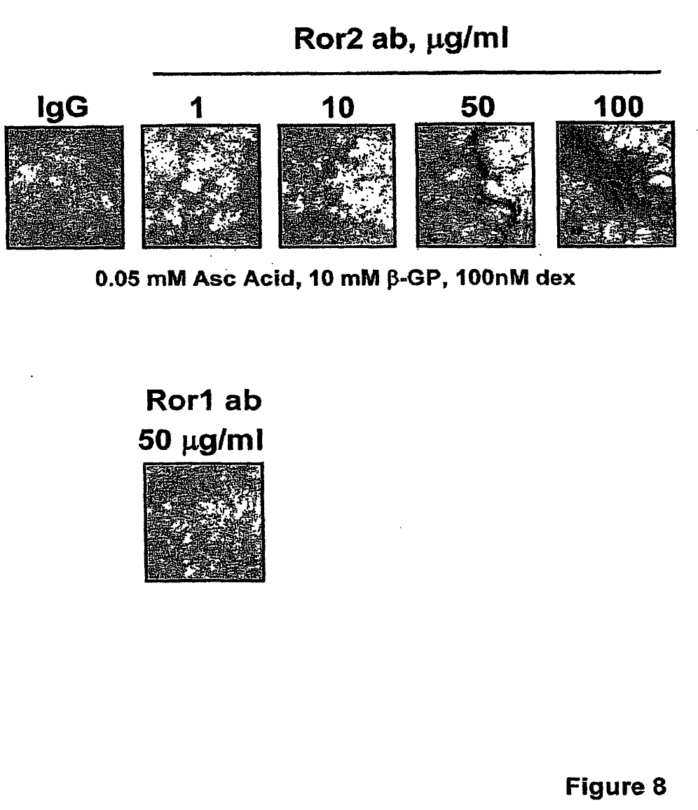

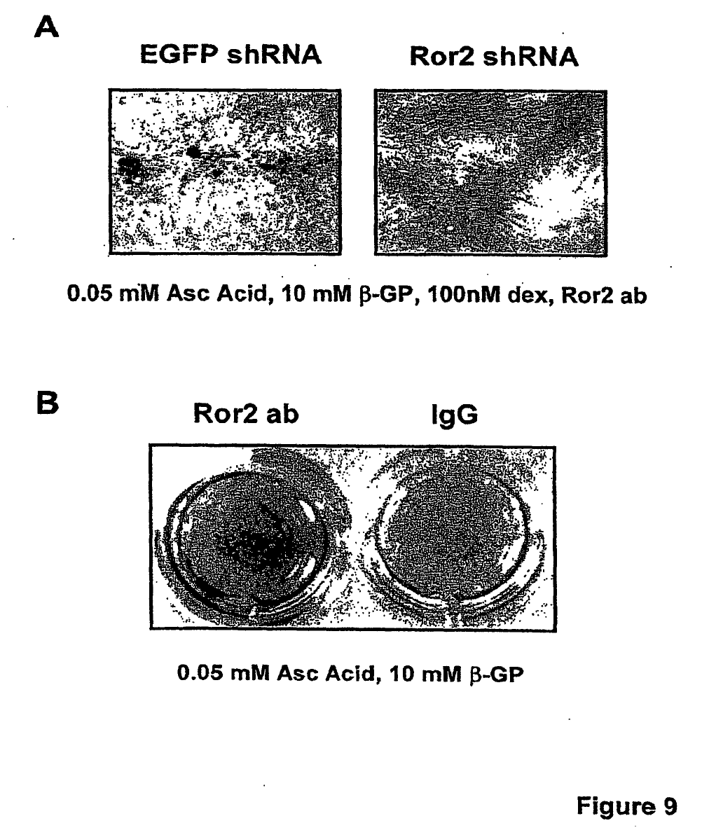

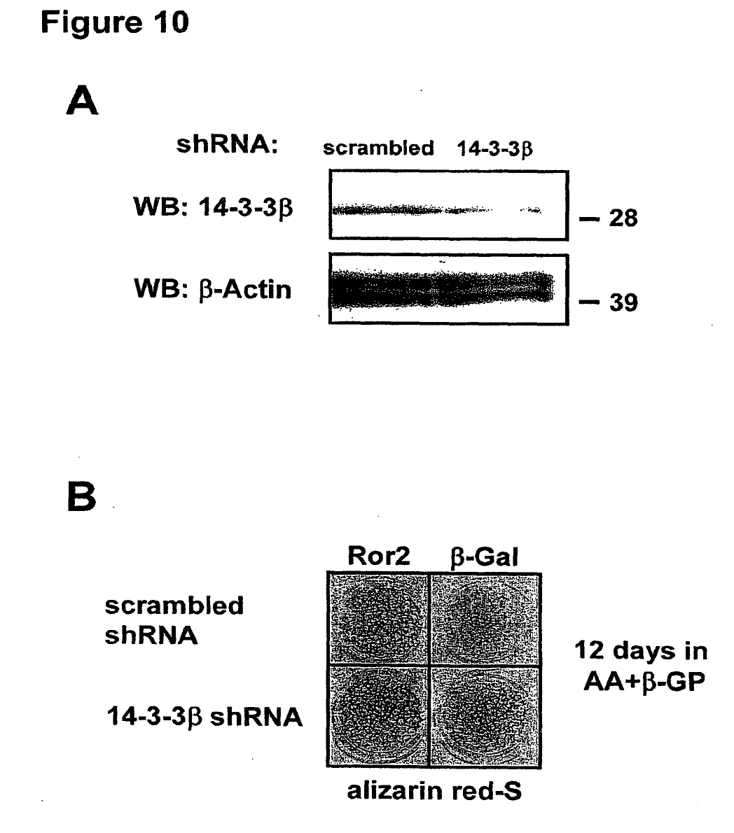

The present invention stems from the discovery of the role of the Ror family and its downstream signaling biomolecules in bone metabolism, particularly osteoblast differentiation. See U.S. patent applications U.S. Ser. No. 10/823,998, 60/463,364, and 60/501,340, each of which is incorporated herein by reference. Applicants have discovered that down-regulating Ror2 expression inhibits dexamethasone-induced osteogenic differentiation of human mesenchymal stem cells (FIG. 1). In contrast, overexpression of Ror2 inhibits the adipogenic differentiation of human mesenchymal stem cells (FIG. 2). Applicants have also shown that down-regulating 14-3-3β expression enhances mineralized matrix formation in human mesenchymal stem cells (FIG. 10). Furthermore, Ror2 overexpression and 14-3-3β inhibition induce greater matrix mineralization than either alone (FIG. 10). Based on these discoveries, agents that modulate Ror2 activity at the protein level or modulate 14-3-3β activity, or pharmaceutical compositions thereof, are useful in the treatment of bone diseases and/or metabolic disorders such as obesity or diabetes. Indeed, an agent that increases the activity of Ror2 protein will promote osteoblast differentiation and thereby increase mineralized bone formation (FIGS. 8 and 9). Also, an agent that inhibits the activity of 14-3-3β will promote osteoblast differentiation and thereby increase mineralized bone formation (FIG. 10).

In one aspect, the invention provides agents that modulate (increase or decrease) the activity of Ror2 protein. In certain embodiments, the agents increase the activity of Ror2 protein. In other embodiments, the agents decrease the activity of Ror2 protein. Typically, these agents work at the protein level increasing or decreasing the activity level of the Ror2 protein. As discussed herein, agents that increase Ror2 activity are useful in promoting mineralized bone formation and osteogenic differentiation. These agents may also be useful in treating obesity by inhibiting adipogenic differentiation (FIG. 2). Without wishing to be bound by any particular theory, increased Ror2 activity seems to promote osteogenic differentiation while inhibiting adipogenic differentiation.

These agents that modulate Ror2 activity may be any type of chemical compound including small molecules, polynucleotides, proteins, peptides, etc. In certain embodiments, the agent is a protein. In other embodiments, the agent is a peptide. In yet other embodiments, the agent is a polynucleotide. In still other embodiments, the agent is a small molecule (e.g., with a molecular weight less than 1500 g/mol). Preferably, the agent is specific for Ror2 protein and does not bind to other biomolecules. In particular, in certain embodiments, the agent does not bind to other Ror family members. In other embodiments, there may be cross-reactivity with other biomolecules or Ror family members; however, the agent's affinity for these other molecules is less than for Ror2 protein.

In certain particular embodiments, the agent acts by causing the dimerization of two Ror2 proteins. The dimerization of Ror2 proteins is thought to lead to the Ror2 receptor's activation. The activation of Ror2 kinase activity leads to the phosphorylation of its binding partners including 14-3-3β protein. Other Ror2 binding partners include, but are not limited to, ADP/ATP carrier protein, UDP-glucose ceramide glucosyltransferase-like 1, 14-3-3 protein gamma, ribophorin I, arginine N-methyltransferase 1, cellular apoptosis susceptibility protein, NOTCH2 protein, and human skeletal muscle LIM-protein 3 (Billiard et al., U.S. patent application U.S. Ser. No. 10/823,998, filed Apr. 14, 2004). The agent typically includes at least two binding domains directed to Ror2 protein. In certain embodiments, the agent has exactly two binding domains directed to Ror2 protein, that is, the agent is bivalent. Other agents that are multivalent are also useful in the present invention. In certain embodiments, the agent is a small molecule or polynucleotide that promotes the dimerization of Ror2 protein. In other embodiments, the agent is a protein or peptide.

In certain embodiments, the agent is an antibody or antibody fragment (e.g., diabody) directed to Ror2 protein. The antibody or antibody fragment may be directed to any region of the Ror2 protein; however, the antigen binding site is preferably not directed to a region which may interfere with Ror2's biological activity (e.g., kinase activity) or interfere with dimerization of the two proteins. The binding of two Ror2 proteins by the antibody or antibody fragment promotes the dimerization of the Ror2 proteins and thereby its activation. The antibody may be polyclonal or monoclonal. The antibody may be of any isotype; however, the IgG isotype is generally preferred. The antibody may be derived from any species; however, for use in humans, the antibody is typically of human origin or has been humanized. If the antibody is to be used in other species, the antibody may be adapted to that species. In certain embodiments, the antibody is a humanized monoclonal antibody. In other embodiments, the antibody is a wholly human antibody. In certain specific embodiments, the antibody is a wholly human monoclonal antibody.

In certain embodiments, an antibody directed to Ror2 protein is prepared by immunizing a mammal such as a rabbit or other rodent with purified human Ror2 protein or peptides derived from Ror2 protein. After immunization, cells producing antibodies such as B-cells or plasma cells are collected and used to prepare hybridomas that are then screened for the production of antibodies directed to Ror2 protein. In certain embodiments, antibodies are screened for their ability to dimerize and/or activate Ror2 protein. Once a B-cell producing the desired antibodies is identified, the B-cell may be immortalized. The resulting hybridoma can then be used to produce the desired monoclonal antibody. The antibody produced by the hybridoma may be further characterized and modified. For example, in certain embodiments, the antibody may be humanized so that administration of the antibody to a human subject does not lead to an adverse reaction, which can range from increased clearance of the therapeutic antibody to fatal anaphylaxis. In certain particular embodiments, the regions of the antibody that recognize Ror2 protein (i.e., the complementarity determining regions) are used to replace the CDRs of a human antibody of different specificity. Techniques for engineering and preparing antibodies are known in the art and are described in U.S. Pat. No. 4,816,567, issued Mar. 28, 1989; U.S. Pat. No. 5,078,998, issued Jan. 7, 1992; U.S. Pat. No. 5,091,513, issued Feb. 25, 1992; U.S. Pat. No. 5,225,539, issued Jul. 6, 1993; U.S. Pat. No. 5,585,089, issued Dec. 17, 1996; U.S. Pat. No. 5,693,761, issued Dec. 2, 1997; U.S. Pat. No. 5,693,762, issued Dec. 2, 1997; U.S. Pat. No. 5,869,619; issued 1991; U.S. Pat. No. 6,180,370, issued Jan. 30, 2001; U.S. Pat. No. 6,548,640, issued Apr. 15, 2003; U.S. Pat. No. 6,881,557, issued Apr. 19, 2005; U.S. Pat. No. 6,982,321, issued Jan. 3, 2006; incorporated herein by reference. In other embodiments, the antibody is evolved and/or modified to achieve an antibody with a higher specificity and/or affinity for Ror2 protein.

In other embodiments, the agent comprises a fragment of an antibody directed to Ror2 protein. One or more fragments of the antibody directed to Ror2 protein may be used. The fragment typically includes the complementarity determining regions (CDRs) responsible for the antibodies affinity for Ror2 protein. In order to dimerize Ror2 protein, at least two binding sites for Ror2 protein are needed; therefore, the agent may be two antibody fragments linked to each other. The fragments may be linked together covalently or non-covalently. For example, the agent may be two F_{ab} fragments covalently linked together. The agent may also be a diabody. In certain embodiments, the agent may include more than two antibody fragments. For example, the agent may include three, four, five, or six antigen binding sites directed to Ror2 protein.

In certain other embodiments, the agent may be a protein, peptide, or small molecule that mimics an antigen binding site of an antibody directed to an Ror protein such as Ror2 protein. These agents may be designed or identified in silico based on the structure of the antigen binding site of the antibody directed to Ror2 protein. The agents may then be tested in various in vitro assays to assess the ability of the agent to dimerize and/or activate Ror2 protein. The agents may also be identified using high-throughput screening methods using libraries of small molecules, peptides, or polynucleotides.

In another aspect, the invention provides methods of using the inventive agents in modulating Ror activity. An agent that modulates the activity of Ror protein, particularly Ror2 protein, is useful for modulating bone-related activity. These agents may also be useful in modulating adipocyte differentiation in the treatment of obesity, diabetes, or other metabolic disorders. There are many diseases and conditions characterized by the need to modulate bone related activity, e.g., enhance bone formation. The most obvious is the case of bone fractures, where it would be desirable to stimulate bone growth and to hasten and complete bone repair. For example, agents that enhance bone formation may be potentially useful in facial reconstruction procedures or ortheopaedic procedures. Other bone deficit conditions include, but are not limited to, bone segmental defects, periodontal disease, metastatic bone disease, osteolytic bone disease, and conditions where connective tissue repair would be beneficial, such as healing or regeneration of cartilage defects or injury. Also of great significance is the condition of osteoporosis, including age-related osteoporosis and osteoporosis associated with post-menopausal hormone status. Other conditions characterized by the need for bone growth include primary and secondary hyperparathyroidism, diabetes-related osteoporosis, disuse osteoporosis, and glucocorticoid-related osteoporosis.

Agents that increase Ror2 activity may be used to promote mineralized bone formation. These agents may also be used to promote osteoblastic differentiation. The promotion of osteoblastic differentiation may be done at the expense of adipogenic differentiation. The agents may also be used to promote mineralized matrix formation.

In another aspect, the invention provides agents that modulate (increase or decrease) the activity of 14-3-3 (e.g., 14-3-3β, 14-3-3γ, etc.). In certain embodiments, the agents inhibit the activity of 14-3-3. In other embodiments, the agents increase the activity of 14-3-3. The agent may work at the nucleic acid or protein level. In certain embodiments, the agent decreases the expression of 14-3-3β. As discussed herein, agents that inhibit 14-3-3β activity are useful in promoting mineralized bone formation and osteogenic differentiation. In certain embodiments, the agent decreases the expression of 14-3-3γ. These agents may also be useful in treating obesity, diabetes, or other metabolic disorders by inhibiting adipogenic differentiation. Without wishing to be bound by any particular theory, down-regulating 14-3-3 expression, particularly 14-3-3β, seems to promote osteogenic differentiation while inhibiting adipogenic differentiation.

These agents that modulate 14-3-3 activity may be any type of chemical compound including small molecules, polynucleotides, proteins, peptides, etc. In certain embodiments, the agent is a protein. In other embodiments, the agent is a peptide. In yet other embodiments, the agent is a polynucleotide. In still other embodiments, the agent is a small molecule. In certain embodiments, the agent is a polynucleotide. In certain embodiments, the agent is a DNA. In other embodiments, the agent is an RNA. In certain embodiments, the agent is a 14-3-3-specific RNAi. In certain particular embodiments, the agent is a 14-3-3β-specific RNAi. In certain particular embodiments, the agent is a 14-3-3-specific siRNA. In certain embodiments, the agents is a 14-3-3β-specific siRNA. In certain particular embodiments, the agent is a 14-3-3-specific shRNA. In certain embodiments, the agent is a 14-3-3β-specific shRNA. In other embodiments, the agent is specific to 14-3-3γ. In particular, in certain embodiments, the agent specifically targets 14-3-3 found in mesenchymal stem cells or bone cells such as osteoblasts. For example, in certain embodiments, the agent includes a targeting moiety. In certain embodiments, the targeting agent is a bisphosphonate or other bone organ targeting agent.

In another aspect, the invention provides methods of using the inventive agents in modulating 14-3-3 activity. An agent that modulates the activity of 14-3-3, particularly 14-3-3β, is useful for modulating bone-related activity. These agents may also be useful in modulating adipocyte differentiation in the treatment of obesity, diabetes, or other metabolic disorders. There are many diseases and conditions characterized by the need to modulate bone-related activity, e.g., enhance bone formation. The most obvious is the case of bone fractures, where it would be desirable to stimulate bone growth and to hasten and complete bone repair. For example, agents that enhance bone formation may be potentially useful in facial reconstruction procedures or orthopaedic procedures. Other bone deficit conditions include, but are not limited to, bone segmental defects, periodontal disease, metastatic bone disease, osteolytic bone disease, and conditions where connective tissue repair would be beneficial, such as healing or regeneration of cartilage defects or injury. Also of great significance is the condition of osteoporosis, including age-related osteoporosis and osteoporosis associated with post-menopausal hormone status. Other conditions characterized by the need for bone growth include primary and secondary hyperparathyroidism, diabetes-related osteoporosis, disuse osteoporosis, and glucocorticoid-related osteoporosis.

Agents that decrease 14-3-3 activity may be used to promote mineralized bone formation. These agents may also be used to promote osteoblastic differentiation. The promotion of osteoblastic differentiation may be done at the expense of adipogenic differentiation. The agents may also be used to promote mineralized matrix formation.

Agents for use in the methods of the invention can be incorporated into pharmaceutical compositions suitable for administration to a subject. As used herein, the agent may be any identified compound (e.g., small, orally active, organic molecules; proteins; immunoglobulins; immunoglobulin fragments; peptides) that modulate Ror molecule (e.g., Ror2 protein) activity or 14-3-3 activity (e.g., 14-3-3β, 14-3-3γ). Such compositions typically comprise the compound and a pharmaceutically acceptable carrier. The compositions of the present invention may contain one or more agents in combination with one or more agents known to modulate bone-related activity. For example, an agent that promotes Ror activity or inhibits 14-3-3 activity may be combined with agents that inhibit bone resorption like estrogens, bisphosphonates, or tissue selective estrogens (i.e., selective estrogen receptor modulators (SERMs)). The inventive agents may be combined with other agents that promote bone formation.

One or more agent is used at a therapeutically effective dose. A therapeutically effective dose refers to that amount of the agent that is sufficient to show a benefit (e.g., a reduction in a sign and/or symptom associated with the disorder, disease, or condition being treated). When applied to an individual ingredient, administered alone, the term refers to that ingredient alone. When applied to a combination, the term refers to combined amounts of the ingredients that result in the benefit, whether administered in combination, serially, or simultaneously. For example, an effective amount for therapeutic uses is the amount of the composition comprising an agent that provides a clinically significant increase in healing rates in fracture repair; reversal of bone loss and prevention of fractures in osteoporotic subjects; reversal of cartilage defects or disorders; prevention or delay of onset of osteoporosis; prevention of further bone loss associated with osteoporosis; stimulation and/or inhibition of bone formation in fracture non-unions and distraction osteogenesis; increase and/or decrease in bone growth into prosthetic devices; repair of dental defects; and the like. Such effective amounts will be determined using routine optimization techniques and are dependent on the particular condition to be treated, the condition of the patient, the route of administration, the formulation, and the judgment of the practitioner and other factors evident to those skilled in the art. The dosage required for the compounds of the invention (for example, in osteoporosis where an increase in bone formation is desired) is the dosage that ensures a statistically significant difference in bone mass between treatment and control groups. This difference in bone mass may be seen, for example, as a 5-20% or more increase in bone mass in the treatment group. Other measurements of clinically significant increases in healing may include, for example, tests for breaking strength and tension, breaking strength and torsion, 4-point bending, increased connectivity in bone biopsies, and other biomechanical tests well known to those skilled in the art. General guidance for treatment regimens may be obtained from experiments carried out in animal models of the disease of interest.

Toxicity and therapeutic efficacy of agents may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents or compounds that exhibit large therapeutic indices are preferred. The data obtained from cell culture assays and animal studies may be used in formulating a range of dosage for use in humans. The dosage of such agents or compounds may be within a range of circulating concentrations that include the ED₅₀ with little or no toxicity. The dosage may vary within this range depending upon the dosage form employed and the route of administration utilized.

For any agent used in the method of the invention, the therapeutically effective dose may be estimated initially from cell culture assays. For example, a dose can be formulated in animal models to achieve a circulating plasma concentration range that includes the ED₅₀ as determined in cell culture or animal studies (i.e., the concentration of the test compound which achieves a half-maximal dimerization of Ror2 protein). Such information can be used to more accurately determine useful doses in humans. Levels in plasma may be measured, for example, by HPLC. The dosage can be chosen by the individual physician in view of the patient's condition. The attending physician would know how to and when to terminate, interrupt, or adjust administration. Conversely, the attending physician would also know to adjust treatment to higher levels if the clinical response were not adequate (precluding toxicity). The magnitude of an administered dose in the management of the disorder of interest will vary with the severity of the condition to be treated. The severity of the condition may, for example, be evaluated, in part, by standard prognostic evaluation methods. Further, the dose and perhaps dose frequency, will also vary according to the age, body weight, and response of the individual patient. A program comparable to that discussed above may be employed in veterinary medicine.

Determination of the proper dosage for a particular situation is within the skill of the art. Generally, treatment is initiated with smaller dosages that are less than the optimum dose of the compound. Thereafter, the dosage may be increased by small increments until the optimum effect under the circumstances is reached. For example, the total daily dosage may be divided and administered in portions during the day, if desired. A daily dosage may be divided in to two, three, or four portions, each of which is administered during a 24 hour period.

In the case of antibodies or antibody fragments as the agent being administered, the agent is typically administered via intravenous infusion. The dosage may range from 1-25 mg/kg every 1-6 weeks. In certain embodiments, the dosage may range from 1-10 mg/kg every 1-6 weeks. In certain embodiments, 1-10 mg/kg of the agent is delivered by intravenous infusion every 3-5 weeks. In other embodiments, 3-6 mg/kg of the agent is delivered by intravenous infusion every 4 weeks.

Depending on the specific conditions being treated, agents may be formulated and administered systemically or locally. A pharmaceutical composition of the invention is formulated to be compatible with its intended route of administration. Techniques for formulation and administration may be found in Remington's Pharmaceutical Sciences, 18th ed., Mack Publishing Co., Easton, Pa. (1990). Suitable routes may include oral, rectal, vaginal, transdermal, transmucosal, or intestinal administration; parenteral delivery, including intramuscular, subcutaneous and intramedullary injections; as well as intrathecal, direct intraventricular, intravenous, intraperitoneal, intranasal, or intraocular injections, just to name a few. Some methods of delivery that may be used include but are not limited to encapsulation in liposomes, incorporation into prosthetic devices, transduction by retroviral vectors, and transfection of cells ex vivo with subsequent reimplantation or administration of the transfected cells.

When the compositions are used pharmaceutically, they are combined with a “pharmaceutically acceptable carrier” for diagnostic and therapeutic use. The formulation of such compositions is well known to persons skilled in this field. Pharmaceutical compositions of the invention may comprise one or more additional agents and, preferably, include a pharmaceutically acceptable carrier.

Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The term “pharmaceutically acceptable carrier” refers to a carrier that does not cause an allergic reaction or other untoward effect in patients to whom it is administered. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of one or more of the agents of the composition. The use of such media and agents for pharmaceutically acceptable substances is well known in the art.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injections include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.), or phosphate buffered saline. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars or polyalcohols such as mannitol, sorbitol, sodium chloride in the composition. Prolonged absorption of the injectable compositions can be brought about by including in the composition an agent which delays absorption, for example, aluminum monostearate and gelatin.

Further, the agents for treating diseases and conditions identified by the present invention may also be co-administered with other therapeutic agents that are selected for their particular usefulness against the condition that is being treated. For example, the agents may be combined with estrogens or estrogen-related compounds or other bone resorption inhibitors. Estrogen compounds include but are not limited to conjugated estrogens, estradiol, and analogs thereof. Other bone-related therapeutic compounds include, but are not limited to, bisphosphonates and related compounds (such as those set forth in U.S. Pat. No. 5,312,814), calcium supplements (Prince, R. L. et al., N. Engl. J. Med. 325, 1189, (1991), vitamin D supplements (Chapuy M. C. et al., N. Engl. J. Med. 327, 1637, (1992), sodium fluoride (Riggs, B. L. et al., N. Engl. J. Med., 327, 620, (1992), androgen (Nagent de Deuxchaisnes, C., in Osteoporosis, a Multi-Disciplinary Problem, Royal Society of Medicine International Congress and Symposium Series No. 55, Academic Press, London, p. 291, (1983), and calcitonin (Christiansen, C., Bone 13 (Suppl. 1):S35, (1992).

In another aspect, the present invention provides a system for identifying agents that activate Ror protein. Methods for determining whether an agent alters the activity of Ror2 protein include performing analyses and assays well known to the skilled artisan. Examples include, but are not limited to, histochemical analysis, western blot analysis, ELISA, enzyme assays (e.g., kinase assays), and functional analyses including, for example, measurements of the extent of Ror or 14-3-3β phosphorylation (higher states of phosphorylation reflecting higher activity). In certain embodiments, the activity of Ror, specifically Ror2 protein, is assessed by determining the phosphorylation status of 14-3-3β, which has been shown to bind Ror2 protein and be phosphorylated by Ror2 protein. The phosphorylation of 14-3-3β protein may be assayed using any techniques known in the art. In particular, immunoprecipitation using anti-phosphotyrosine antibodies may be used to follow the phosphorylation of 14-3-3β protein. Alternatively, radioactive isotopes of phosphorus (e.g., ³²P-γ-ATP) may also be used.

The present invention also provides a method for identifying agents that modulate bone-related activity, wherein an increase or decrease in the activity of an Ror molecule (e.g., Ror2 protein) indicates that the agent modulates bone-related activity.

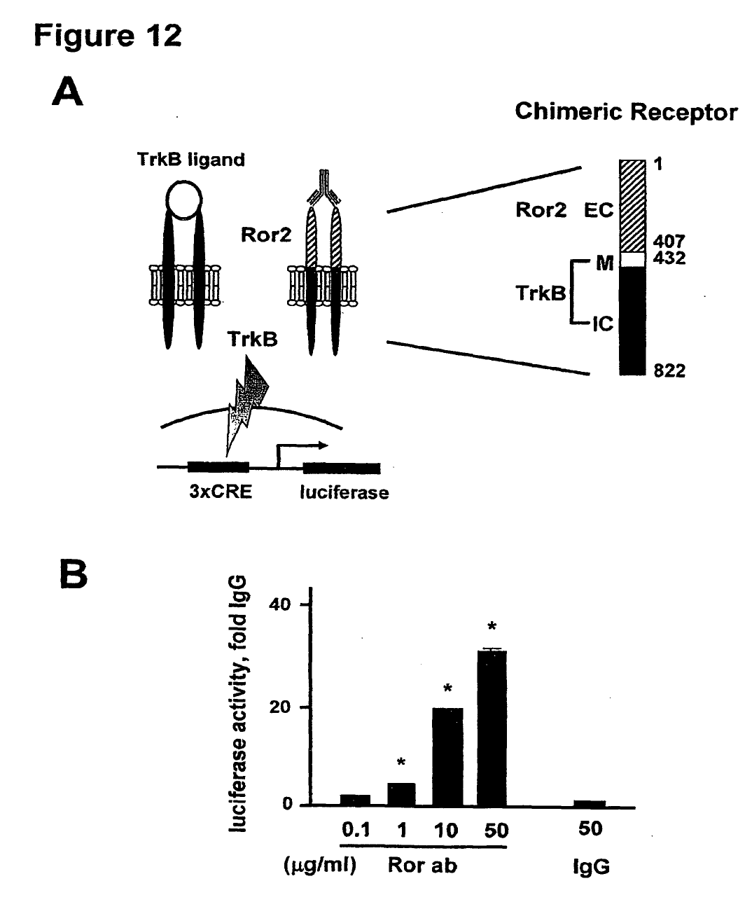

In certain embodiments, the present invention provides an assay method for identifying agents that promote the dimerization of Ror2 using a chimeric receptor (e.g., Ror2/TrkB) and a reporter gene, such as luciferase, regulated by the dimerization of Ror2. In certain embodiments, a cell expressing a chimeric receptor comprising the extracellular domain of Ror2 fused to the intracellular domain of TrkB is used in the inventive assay. In certain particular embodiments, amino acids 1-407 of the extracellular domain of Ror2 protein are fused to the transmembrane and intracellular domains of TrkB (amino acids 432-822). In other embodiments, a different intracellular domain is used in constructing the chimera. For example, any intracellular domain that is activated upon dimerization could be used in place of the TrkB domain. Preferably, the intracellular domain is from a single-span transmembrane receptor, and the signalling pathway is known. Non-limiting examples of other intracellular domains that could be used in preparing the chimeric receptor include the intracellular domain of TrkA, TrkC, EGFR, PDGFR, and FGFR. The intracellular domain is activated upon dimerization of the extracellular domain and turns on a signalling cascade that eventually leads to the up-regulation of a report gene. For example, agents that dimerize the extracellular Ror2 domains of the chimeric receptor cause the activation of the TrkB signaling pathway in the case of the Ror2/TrkB chimera. Activation of the TrkB signaling pathway is assessed by the use of a cAMP response element (CRE) promoter-reporter gene system. Activation of another signaling pathway such as EGFR would require another reporter gene system such as one based on STAT binding elements, which is turned on by the EGFR pathway. Activation of the TrkB pathway causes the stimulation of the CRE promoter which in turn increases the expression of any reporter gene under its control. Easily assayed reporters such as luciferase (LUC), green fluorescent protein (GFP), β-galactosidase (GAL), β-glucuronidase (GUS), chloramphenicol acetyltransferase (CAT), etc. may be placed under the control of the CRE promoter and used in the inventive assay. In certain embodiments, luciferase is used as the reporter gene. In other embodiments, green fluorescent protein is used as the reporter gene. In certain embodiments, the CRE promoter-reporter construct on a plasmid is transfected into the cell expressing the chimeric receptor. In other embodiments, the construct is part of the genome of the cell. In certain embodiments, the construct is stably transfected into the cell. The inventive assay system based on the Ror2/TrkB chimera has been validated using an Ror2-specific antibody shown herein to dimerize Ror2. The Ror2-specific antibody causes a dose-dependent increase in the observed reporter (i.e., luciferase) activity. See, FIG. 12.

The inventive chimeric receptor assay system may be modified using different intracellular domains paired with a corresponding promoter system. Examples of other intracellular domains include the intracellular domain of TrkA, TrkC, EGFR, PDGFR, and FGFR. A corresponding promoter modulated by the intracellular domain would then be used in the reporter system. For example, a STAT binding element could be used in a system using a chimeric receptor with the EGFR intracellular domain.

The present invention includes kits for performing the inventive chimeric receptor assay. These kits include some or all of the components necessary to screen test agents using the inventive assay. In certain embodiments, the components of the kit are conveniently packaged for use by a researcher. The kits may include any or all of the following: DNA constructs, cell lines, buffers, enzymes, multi-well plates, positive and negative controls, media, antibiotics, nucleotides, instructions, etc. In certain embodiments, the kit includes a cell line expressing the Ror2/TrkB chimeric receptor. In other embodiments, the kit includes a DNA construct encoding the Ror2/TrkB chimeric receptor. In other embodiments, the kit includes a reporter gene operably linked to the CRE promoter. In certain embodiments, the kit includes a luciferase gene operably linked to the CRE promoter. The reporter gene/CRE promoter construct may be a plasmid.

The present invention includes the chimeric receptor with the extracellular Ror2 domain used in the inventive assay described above. An exemplary amino acid sequence of a chimeric Ror2/TrkB receptor is as follows. The amino acid sequence derived from the Ror2 protein is shown in upper case letters; the amino acid sequence derived from the TrkB protein is shown in lower case letters.

(SEQ ID NO: 4)

MARGSALPRRPLLCIPAVWAAAALLLSVSRTSGEVEVLDPNDPLGPLDGQ
DGPIPTLKGYFLNFLEPVNNITIVQGQTAILHCKVAGNPPPNVRWLKNDA
PVVQEPRRIIIRKTEYGSRLRIQDLDTTDTGYYQCVATNGMKTITATGVL
FVRLGPTHSPNHNFQDDYHEDGFCQPYRGIACARFIGNRTIYVDSLQMQG
EIENRITAAFTMIGTSTHLSDQCSQFAIPSFCHFVFPLCDARSRAPKPRE
LCRDECEVLESDLCRQEYTIARSNPLILMRLQLPKCEALPMPESPDAANC
MRIGIPAERLGRYHQCYNGSGMDYRGTASTTKSGHQCQPWALQHPHSHHL
SSTDFPELGGGHAYCRNPGGQMEGPWCFTQNKNVRMELCDVPSCSPRDSS
KMGILYlsvyavvviasvvgfcllvmlfllklarhskfgmkgpasvisnd
ddsasplhhisngsntpssseggpdaviigmtkipvienpqyfgitnsql
kpdtfvqhikrhnivlkrelgegafgkvflaecynlcpeqdkilvavktl
kdasdnarkdfhreaelltnlqhehivkfygvcvegdplimvfeymkhgd
lnkflrahgpdavlmaegnppteltqsqmlhiaqqiaagmvylasqhfvh
rdlatrnclvgenllvkigdfgmsrdvystdyyrvgghtmlpirwmppes
imyrkfttesdvwslgvvlweiftygkqpwyqlsnneviecitqgrvlqr
prtcpqevyelmlgcwqrephmrknikgihtllqnlakaspvyldilg

As would be appreciated by one of skill in this art, various mutations, deletions, substitutions, etc. may be made in the inventive chimeric protein without departing from the invention. In certain embodiments, the chimeric protein is at least 99%, 98%, 95%, 90%, 80%, or 70% homologous to amino acid sequence above. In certain embodiments, the chimeric receptor activates a signaling pathway such as the TrkB pathway upon dimerization, which is caused by dimerization of the extracellular Ror2 domains of the chimeric receptor. As would be appreciate by those of skill in this art, various changes to the protein sequence above may be made without changing the activity of the receptor. These variants of the chimeric receptor are considered to be within the scope of the invention. The present invention also includes polynucleotide sequences that encode the chimeric receptor or variants thereof. The coding sequence is optionally operably linked to a promoter, enhancers, regulatory elements, etc. that modulate the expression and/or translation of the chimeric protein. The present invention also includes cells that include the inventive polynucleotide sequence encoding the chimeric receptor.

The methods of the present invention may be modified or performed in any available format, including high throughput assays. High throughput assays are useful for screening a large number of test agents in a given period of time. In another embodiment, assays using cell-based screening are performed. U.S. Pat. No. 6,103,479, issued Aug. 15, 2000, incorporated herein by reference, discloses miniature cell array methods and apparatus for cell-based screening. Methods have been described for making uniform micro-patterned arrays of cells for other applications, for example photochemical resist-photolithography (Mrksich and Whitesides, Ann. Rev. Biophys. Biomol. Struct., 25, 55-78, (1996)). U.S. Pat. No. 6,096,509, issued Aug. 1, 2000, incorporated herein by reference, provides an apparatus and method for real-time measurement of a cellular response to a test agent on a flowing suspension of cells, in which a homogeneous suspension of each member of a series of cell types is combined with a test compound at a specific concentration, directed through a detection zone, and a cellular response of the living cells is measured in real time as the cells in the test mixture are flowing through the detection zone. The patent discloses the use of the apparatus in automated screening of libraries of test agents (e.g., small molecules). The methods disclosed in these U.S. patents can be modified to determine whether test agents modulate the expression or activity of Ror molecule using cells such as osteoblastic cells (primary osteoblasts, human osteoblastic cells such as TE-85, U2OS, SaOS-2 or HOB, rat osteoblastic cells such as UMR 106 or ROS 17/2.8, mouse osteoblastic cells such as MC3T3, or others), non-osteoblastic cells (COS-7 and others), stem cells (mesenchymal stem cells, embryonic stem cells), progenitor cells, or engineered cells containing Ror nucleotide sequences. In yet other embodiments, assays based on enzyme assays (e.g., kinase assays) are performed.

Test agents identified as useful in modulating the activity of Ror protein may then be further tested. In certain embodiments, the agents are tested in other cell-based assays or non-cell based assays. The compounds may be tested in animal models of various diseases including animal models of various bone disease and disorders. For example, agents may be tested in animal models of bone fractures, osteoporosis, bone cancers, bone loss, etc.

The present invention incorporates by reference methods and techniques well known in the field of molecular and cellular biology. These techniques include, but are not limited to techniques described in the following publications: Old, R. W. & S. B. Primrose, Principles of Gene Manipulation: An Introduction To Genetic Engineering (3d Ed. 1985) Blackwell Scientific Publications, Boston. Studies in Microbiology; V. 2:409 pp. (ISBN 0-632-01318-4), Sambrook, J. et al. eds., Molecular Cloning: A Laboratory Manual (2d Ed. 1989) Cold Spring Harbor Laboratory Press, NY. Vols. 1-3. (ISBN 0-87969-309-6), Miller, J. H. & M. P. Calos eds., Gene Transfer Vectors For Mammalian Cells (1987) Cold Spring Harbor Laboratory Press, NY. 169 pp. (ISBN 0-87969-198-0).

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion, embodiments, and these examples, one skilled in the art will readily appreciate that many modifications are possible in the exemplary embodiments without materially departing from the novel teachings of this inventions, and without departing from the spirit and scope thereof. Furthermore, one can make various changes to and modifications of the invention to adapt it to various usages and conditions. Accordingly, all such modifications are intended to be included within the scope of this invention as defined in the following claims.

The patents, applications, test methods, and publications mentioned herein are hereby incorporated by reference in their entirety.

### General Methods

### Materials and Tissue Culture

Except where noted, tissue culture reagents were purchased from Invitrogen Corporation (Carlsbad, Calif.); other reagents and chemicals were purchased from either Sigma Chemical Co. (St. Louis, Mo.) or Invitrogen. GST-tagged cytosolic domain of recombinant human Ror2 was obtained from Invitrogen and GST-tagged recombinant human 14-3-3β was from Biomol International, LP (Plymouth Meeting, Pa.). Anti-Flag M2 mouse monoclonal antibody, anti-Flag M2 affinity agarose, and anti-β-actin mouse monoclonal antibody were obtained from Sigma; anti-human Ror2 goat polyclonal antibody was purchased from R&D Systems (Minneapolis, Minn.); anti-14-3-3β and anti-His rabbit polyclonal antibodies were from Santa Cruz Biotechnology, Inc. (Santa Cruz, Calif.). Unconjugated and agarose-conjugated anti-phosphotyrosine antibody (4G10) were obtained from Upstate Cell Signaling Solutions (Charlottesville, Va.); immobilized phosphotyrosine antibody P-Tyr-100 was from Cell Signaling Technologies (Beverly, Mass.); and protein A sepharose and glutathione sepharose were purchased from Amersham Biosciences (Buckinghamshire, England). Horseradish peroxidase (HRP)-conjugated secondary antibodies were from Santa Cruz Biotechnology.

The human mesenchymal stem cells (hMSC) were purchased from Cambrex, Inc. (Baltimore Md.) and maintained at 37° C. in a 5% CO₂-95% humidified air incubator using hMSC growth medium (MSCGM, Cambrex). The U2OS human osteosarcoma cells were kept at 37° C. in McCoy's 5A Modified Medium, containing 10% heat-inactivated fetal bovine serum (FBS), 1% penicillin-streptomycin, and 2 mM glutaMAX-I.

### Plasmids and Adenoviruses

Generation of the human Ror2-Flag expression plasmid has been previously described (Billiard and Bodine, U.S. patent application U.S. Ser. No. 10/823,998, filed Apr. 14, 2004; Billiard et al. Mol Endo 19, 90-101, 2005). The Ror2-His construct was generated by replacing the Flag epitope tag at the COOH terminal of the Ror2-Flag with the sequence coding for 6 histidines. The GST fusion of the cytosolic domain of Ror2 (GST-Ror2c) was obtained by inserting the intracellular domain of the human Ror2 (coding for amino acids 428-944) in frame following the GST tag in the pGEX-4T-2 vector (Amersham).

Full-length human 14-3-3β cDNA was purchased from Open Biosystems (Huntsville, Ala.) and subcloned into pET28a bacterial expression vector.

Adenoviruses containing human coxsackie adenovirus receptor (hCAR), Ror2-specific shRNA and EGFP-specific shRNA were obtained from Galapagos, Inc. (Mechelen, Belgium). Generation of Ror2, Ror2KD and β-galactosidase (β-gal) adenoviruses has been described (Billiard and Bodine, U.S. Application U.S. Ser. No. 10/823,998, filed Apr. 14, 2004, incorporated herein by reference).

### Calvarial Organ Culture and Infection

Calvariae were excised from 4 days old mouse littermates, cut along the sagittal suture and incubated for 24 h in serum-free BGJ medium containing 0.1% BSA. Each half of the calvaria was then placed with the concave surface downward on a stainless steel grid (Small Parts Inc, Miami, Fla.) in a well of a 12-well plate. Each well contained 1 ml of BGJ medium with 1% FBS, without or with β-gal or Ror2 adenoviruses (3.75 mln viral particles/well). Calvariae were incubated in a 5% CO₂-95% humidified air incubator and the medium and adenoviruses were changed after 4 days.

After 7 days of incubation in presence of adenovirus, calvariae were fixed in 10% neutral phosphate-buffered formaldehyde at RT for 72 hours, then decalcified for 6 hours in 10% EDTA in PBS. Calvariae in each group were embedded in parallel in the same paraffin block, and 4 μm sections were stained with hematoxylin-eosin. Consistent bone areas (200 μm away from frontal sutures) were selected for histomorphometric analysis. In brief, a 200 μm square grid was placed on each calvaria and the number of osteoblasts and total bone area were determined with the Osteomeasure System (Osteometrics Inc, Atlanta, Ga.). All cells on the bone surface were counted as osteoblasts. Medium calcium was measured using Calcium Diagnostics Kit (Sigma) according to manufacturer's protocol

### Viral Infection

Human MSC were seeded at 6,000/cm² in 12-, or 6-well plates and allowed to adhere and proliferate overnight. Cells were infected for 24 h in 0.4 ml/cm² MSCGM using Ror2, Ror2KD, or β-gal adenoviruses at multiplicity of infection (MOI)=750 in presence of hCAR (MOI=750) to improve infection efficiency. After 24 h, cells were washed once in PBS and MSCGM, MSCGM supplemented with 0.05 mM ascorbic acid and 10 mM β-glycerophosphate, or MSCGM containing adipogenic supplements (PT-3004, Cambrex) were added. Where indicated, 100 nM dexamethasone (dex) and/or the indicated antibodies were added to the medium. For shRNA infection, cells were seeded at 6,000/cm² in 12- or 6-well plates and allowed to adhere and proliferate for 3 days. Cells were infected for 72 h in 0.4 ml/cm² MSCGM using adenoviruses coding for Ror2-specific shRNA or EGFP-specific shRNA at 4,000 viral particles per cell (based on the original seeding density) in presence of hCAR (MOI=750). After 72 h, cells were washed once in PBS, and MSCGM or MSCGM supplemented with 0.05 mM ascorbic acid, 10 mM β-glycerophosphate were added. Where indicated 100 nM dex and/or specific antibodies were added. Every 5 days, either the entire medium or ½ of it was replaced with fresh medium.

U2OS cells were seeded at 75,000/cm² in 6-well plates and infected 24 h later with Ror2, Ror2KD, or β-gal adenovirus at MOI=100. Infection was allowed to proceed for 24 h and cell extracts were collected another 24 h later.

### Alizarin Red-S Histochemical Staining

Formation of mineralized nodules by hMSC was determined on 12-well plates by alizarin red-S histochemical staining. The cells and matrix were fixed at RT for 1 h with 70% (v/v) ethanol, washed with de-ionized water and stained for 10 min at RT with 40 mM alizarin red-S, pH 4.2. The stained matrix was washed with de-ionized water and photographed. To quantify the level of alizarin red-S staining, the dye was eluted with 1 ml/well of 10% (w/v) Cetylpyridinium Chloride. Alizarin red-S in the eluted samples was quantified (versus a standard curve of 0-800 μM dye) at 562 nm with a microplate reader.

### Oil Red O Histochemical Staining

Adipogenesis was monitored in hMSC on 12-well plates by Oil red 0 histochemical staining. The cells were fixed at RT for 2 h with 10% neutral buffered formalin, washed with PBS and stained for 10 min at RT with 18 mg/mL Oil red 0 in 60% isopropanol, pH 7. The stained cells were washed with PBS and photographed.

### RNA Isolation and Real-Time PCR Analysis

Total cellular RNA was isolated using the RNeasy Kit (Qiagen, Valencia, Calif.) following the manufacturer's instructions and subjected to real-time RT-PCR analysis using the ABI PRISM 7700 Sequence Detection System (Applied Biosystems, Foster City, Calif.). All mRNA levels were normalized to the levels of a housekeeping gene, cyclophilin B. Primers and probes for human C/EBPα and PPARγ were purchased from Applied Biosystems; primers and probe for human cyclophilin B were as follows: 5′-CACCAACGGCTCCCAGTT-3′ (forward primer, 438-455, SEQ ID NO: 1), 5′-AACACCACATGCTTGCCATCT-3′ (reverse primer, 486-506, SEQ ID NO: 2), and 5′-TTCATCACGACAGTCAAGACAGCCTGG-3′ (probe, 457-483, SEQ ID NO: 3).

### Transient Transfections

For Ror2 plasmid transfections, U2OS cells were seeded at ˜80% confluent density and transfected 24 h later with 11 μg of total plasmid DNA per 19.6 cm² using Fugene6 transfection reagent (Roche Applied Science, Indianapolis, Ind.) per manufacturer's instructions. For siRNA transfections, U2OS cells were plated on 6-well plates at 52,000 cells/cm² and transfected 24 h later with 25 nM of Ror2 siRNA or non-specific siRNA (both from Dharmacon Inc., Lafayette, Colo.) using 10 μl of Lipofectamine 2000 reagent per manufacturer's instructions.

### Immunoprecipitation and Western Immunoblotting

Cells were solubilized in lysis buffer (150 mM NaCl, 50 mM Tris-HCl, pH 7.5, 1 mM EDTA, 1% Triton X100) supplemented with protease and phosphatase inhibitor cocktails (Sigma) and the extracts were clarified by centrifugation at 10,000×g for 10 min at 4° C. For Flag immunoprecipitation, 1 mg of total cell lysates was incubated with 30 μl of M2 Flag affinity agarose (Sigma) for 1 h with rotation at 4° C. The beads were collected by centrifugation and washed three times in lysis buffer containing 350 mM NaCl and three times in lysis buffer. For 14-3-3β precipitation, 15 μl of 14-3-3β antibody was incubated with 30 μl of protein A sepharose in 1 ml of lysis buffer overnight at 4° C. and the beads were collected by centrifugation and washed in lysis buffer prior to addition of 1 mg of total cell lysates. The binding reaction was carried out for 2 h at 4° C. with mild rotation and the beads were collected and washed as for Flag precipitation. For phosphotyrosine precipitation, 1 mg (for detecting over-expressed proteins) or 1.5 mg (for detecting endogenous proteins) of cellular extract was added to 100 μl of G410 beads and allowed to attach for 3 h at 4° C. At this time, P-Tyr-100 immobilized antibody (100 μl) was added to the mix for an additional 3 h. All beads were collected by centrifugation and washed as for Flag precipitation. At the end of all immunoprecipitation reactions, the beads were boiled in 30-50 μl of 2xLDS-PAGE buffer with reducing agent (Invitrogen), and the solubilized proteins were separated by SDS-PAGE. The gels were transferred onto 0.45 μm nitrocellulose membrane before detection with each specific antibody.

For immunoblotting without precipitation, the indicated amounts of total cellular lysates were resolved by SDS-PAGE under denaturing and reducing conditions before transfer onto 0.45 μm nitrocellulose membranes and detection with each specific antibody.

### GST Pool-Down and In Vitro Kinase Assay

14-3-3β was in vitro translated from 14-3-3β-pET28a using Expressway™ in vitro protein synthesis system (Invitrogen) per manufacturer's instructions in a 50 μl reaction. GST-Ror2c in pGEX-4T-2 or pGEX-4T-2 (coding for GST alone) were transformed into BL21(DE3) strain of Escherichia coli. Cultures were grown to an A₆₀₀ of 0.7 and induced to express recombinant proteins by addition of isopropyl-1-thio-β-D-galactopyranoside (Sigma; final concentration of 1 mM) and incubation for 4 h. Bacterial pellets were harvested by centrifugation, washed in PBS, and resuspended in 30 ml of PBS supplemented with protease and phosphatase inhibitor cocktails (Sigma). Cells were lysed by passaging twice through a French Pressure Cell Press (Spectronic Instruments, Rochester, N.Y.) at 16,000 p.s.i., and bacterial debris was removed by centrifugation. The produced GST-Ror2c or GST proteins were incubated with glutathione sepharose for 4 h at 4° C. The beads were washed, resuspended in 1 ml PBS and the entire 50 μl of 14-3-3β in vitro translation reaction was added for 4 h at 4° C. At the end of this incubation, the beads were washed three times in PBS, boiled in 2xLDS-PAGE buffer with reducing agent (Invitrogen), and the solubilized proteins were separated by SDS-PAGE. The gels were transferred onto 0.45 μm nitrocellulose membrane before detection with each specific antibody.

For the in vitro kinase assay, 6.5 μg of purified recombinant human GST-14-3-3β (Biomol) was resuspended in 25 μl of kinase reaction buffer (10 mM MgCl₂, 50 mM Tris-HCl pH 7.5, 1 mM dithiotriethol (DTT), 1 mM ATP) with or without addition of 0.9 μg of purified recombinant human GST-R2c (Invitrogen). The kinase reaction was allowed to proceed for 30 min at 37° C. and stopped by boiling in 1xLDS buffer with reducing agent (Invitrogen). Proteins were resolved by SDS-PAGE, transferred onto 0.45 μm nitrocellulose membranes and detected with phosphotyrosine antibody. Subsequently, the membrane was stripped and reprobed with 14-3-3β antibody to verify equal loading.

### Statistical Analysis

Data are presented as means±SE. Statistical significance was determined using one-way ANOVA or Student's t test. Results were considered statistically different when P<0.05.

### Example 1

### Endogenous Ror2 Plays a Role in hMSC Differentiation

We have previously shown that Ror2 expression increases during osteogenic differentiation of hMSC (Billiard et al, U.S. patent application U.S. Ser. No. 10/823,998, filed Apr. 14, 2004; Billiard et al. Mol Endo 19, 90-101, 2005; each of which is incorporated herein by reference). To assess if the rise in Ror2 expression during hMSC differentiation is critical for osteoblastogenesis, we performed dex-induced differentiation when Ror2 expression was inhibited. To this end, hMSC were infected with the adenovirus containing Ror2-specific shRNA, which indeed strongly inhibited the dex-induced rise in Ror2 protein expression when compared to EGFP-specific control shRNA (FIG. 1A). Infection with Ror2 shRNA almost completely abrogated the ability of dex to induce matrix mineralization (FIGS. 1B and C), suggesting that the rise in Ror2 at least in part mediates dex-induced osteoblastic differentiation of hMSC.

### Example 2

### Ror2 Over-Expression Suppresses Adipogenic Differentiation of hMSC

We have also previously shown that Ror2 over-expression initiates commitment of MSC to osteoblastic lineage as well as promotes differentiation at both early and late stages of osteoblastogenesis (Billiard et al., U.S. patent application U.S. Ser. No. 10/823,998, filed Apr. 14, 2004; incorporated herein by reference). We now assessed effects of Ror2 on the alternate fate of hMSC, adipogenesis, induced by incubation in indomethacin- and IBMX-containing adipogenic cocktail. Human MSC were infected with adenoviruses coding for the wild-type Ror2 or the kinase domain mutant (Ror2KD), each containing a COOH-terminal flag epitope tag. In the Ror2KD, three lysines at positions 504 (in the putative ATP biding domain), 507, and 509 were replaced with isoleucines resulting in a significantly reduced tyrosine kinase activity (Hikasa et al. Development 129, 5227-5239, 2002; Billiard et al., U.S. patent application U.S. Ser. No. 10/823,998, filed Apr. 14, 2004; Billiard et al. Mol Endo 19, 90-101, 2005). For control, hMSC were infected with β-galactosidase (β-gal) expression cassette in the same adenoviral background. Both Ror2 and Ror2KD mutant inhibited expression of the major adipogenic transcription factors, CCAAT/enhancer-binding protein a (C/EBPα) and peroxisome proliferator-activated receptor γ (PPARγ) (FIG. 2A), and caused marked decrease in the ability of hMSC to form Oil red O-positive lipid-producing adipocytes (FIG. 2B). Taken together with our previous results (Billiard et al., U.S. patent application U.S. Ser. No. 10/823,998, filed Apr. 14, 2004; incorporated herein by reference), these data indicate that Ror2 alters cell fate of MSC by shifting the balance of transcription factors to favor osteoblastogenesis.

### Example 3

### Ror2 Increases Total Bone Area of Mouse Calvariae

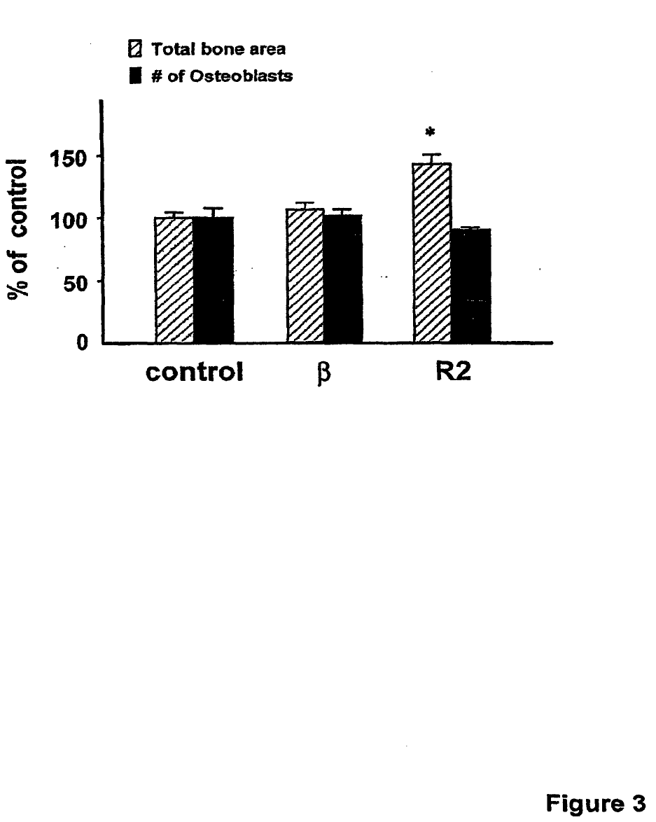

We next tested if in vitro effects of Ror2 on hMSC differentiation translate into increased bone formation in ex vivo organ cultures. Calvarial bones of 4 days old mouse littermates were left uninfected or infected with β-gal or Ror2 adenoviruses. After 7 days of culture in presence of adenovirus, the bones were stained with hematoxylin-eosin and consistent 200 μm² sections (200 μm away from frontal sutures) were subjected to histomorphometric analysis using Osteomeasure System. Under control, uninfected conditions, 200 μm² section of calvaria contained 5171±235 μm² of bone area and 84±6.5 osteoblasts. Infection with Ror2 virus caused 50% increase in the total bone area without affecting the osteoblast number indicating that Ror2 activates osteoblasts to produce more bone matrix (FIG. 3).

### Example 4

### 14-3-3β is the first identified substrate of the Ror2 Kinase

We have previously reported identification of 9 potential Ror2 binding factors in U2OS osteosarcoma cells by mass spectroscopy (Billiard et al., U.S. patent application U.S. Ser. No. 10/823,998, filed Apr. 14, 2004; incorporated herein by reference). Of these, 14-3-3 proteins have been shown to play a role in cell cycle progression and differentiation (Mackintosh, Biochem J 381, 329-342, 2004) and we selected 14-3-3β, for follow-up studies. We first confirmed the interaction observed by mass spectroscopy using immunoprecipitation techniques.

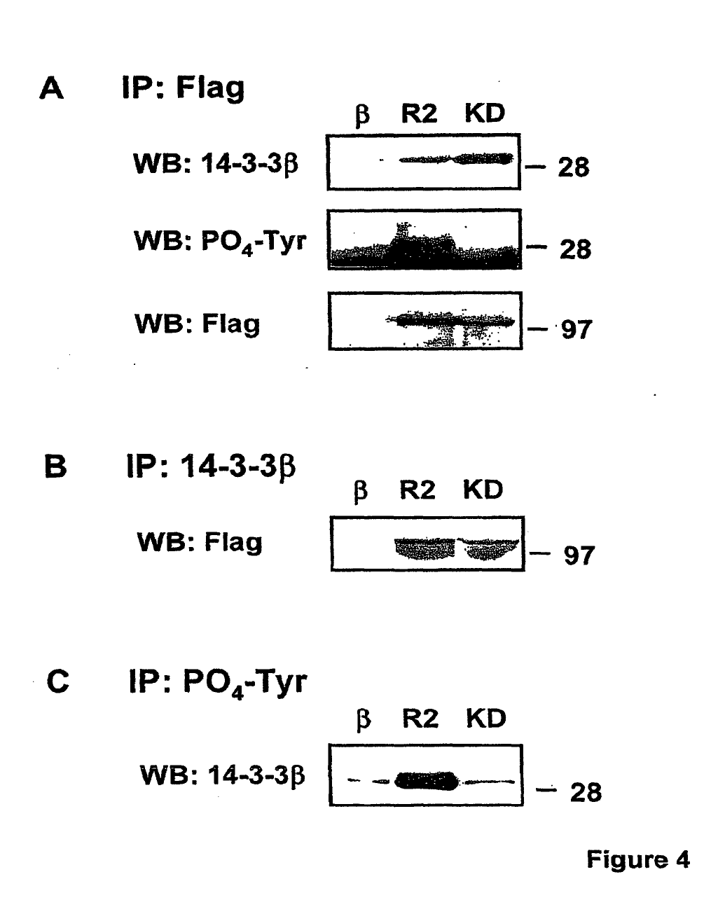

U2OS cells were infected with β-gal, Ror2, or Ror2KD adenovirus and total cellular proteins were isolated and subjected to immunoprecipitation on anti-Flag affinity agarose followed by immunoblotting with anti-14-3-3β antibody (FIG. 4A, top panel). Very low amount of 14-3-3β was precipitated under control conditions (β-gal-infected cells), but a significant amount co-precipitated upon Ror2 over-expression. Complex formation was even stronger with the Ror2KD mutant indicating that the kinase activity leads to complex dissociation. Equal levels of precipitation were verified by immunoblotting with anti-Flag antibody (FIG. 4A, bottom panel). The interaction between 14-3-3β and Ror2 was further confirmed by precipitating 14-3-3β on 14-3-3β-specific antibody and verifying the presence of Ror2 in the complex by anti-Flag immunoblotting (FIG. 4B).

To assess if Ror2 causes phosphorylation of 14-3-3β, we re-probed the blot in FIG. 4A with anti-phosphotyrosine antibody. This antibody identified a phosphorylated protein that migrated at the same molecular weight as 14-3-3β and was present only in cells expressing the wild-type Ror2, but not β-gal or the kinase-inactive mutant (FIG. 4A, middle panel). This suggests that Ror2, directly or indirectly, phosphorylates 14-3-3β on tyrosine residue(s). This hypothesis was confirmed by immunoprecipitating all tyrosine phosphorylated proteins in U2OS extracts on anti-phosphotyrosine antibody and observing a significant increase in the amount of phospho-14-3-3 β upon Ror2 over-expression (FIG. 4C).

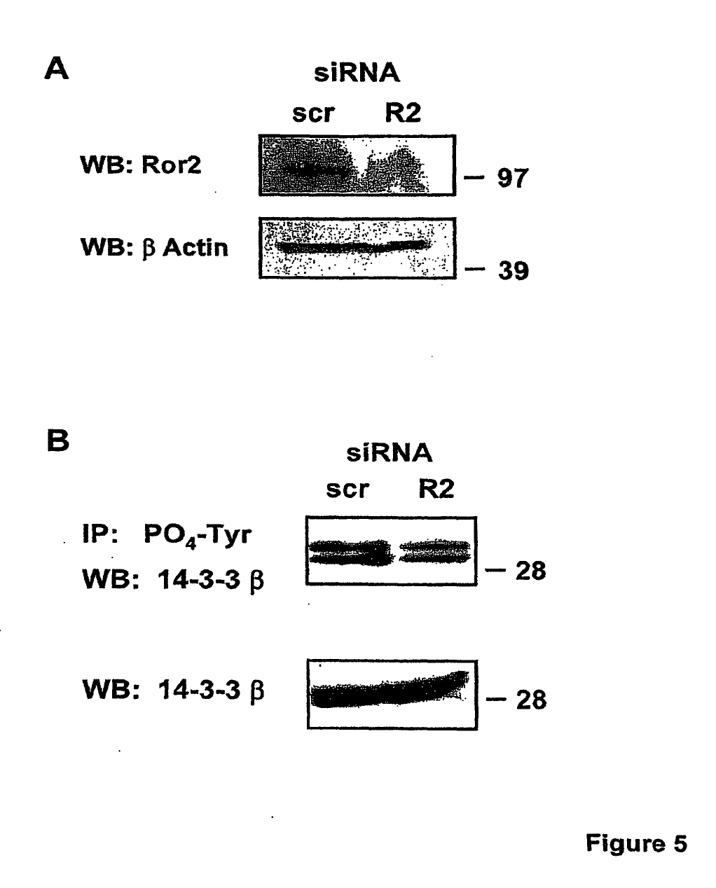

To test if endogenous Ror2 mediates the background phosphorylation of 14-3-3β observed in FIG. 4C, we inhibited Ror2 expression in U2OS cells by Ror2-specific siRNA. As shown in FIG. 5A, transfection with Ror2-specific siRNA resulted in almost complete inhibition of Ror2 protein expression when compared to scramble control siRNA. The decrease in Ror2 expression had no effect on the amount of 14-3-3β protein in U2OS cells, but caused significant down-regulation of its tyrosine phosphorylation (FIG. 5B). The apparent increase in the extent of background phosphorylation of 14-3-3β compared to FIG. 4C results from a longer exposure time used here.

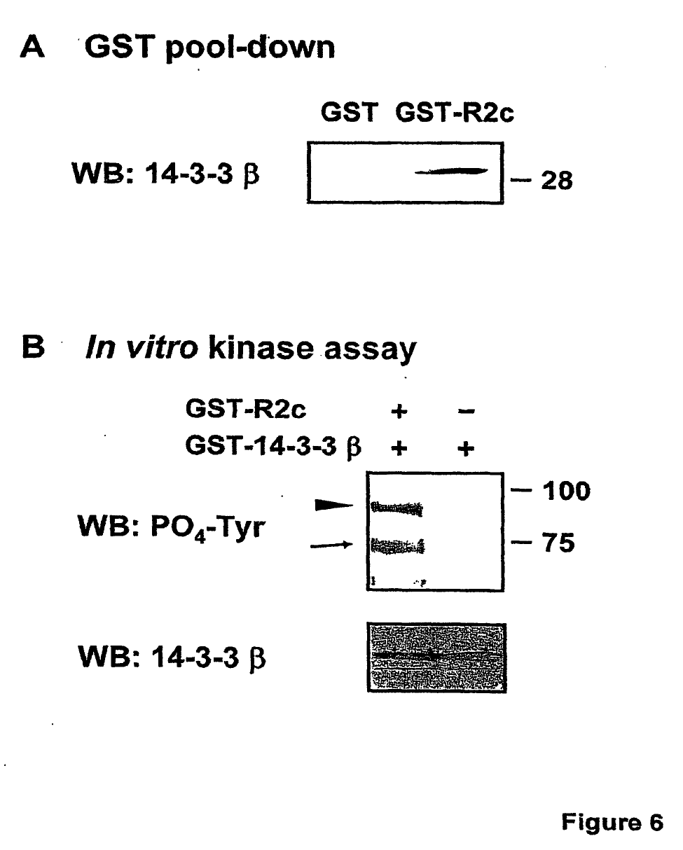

To assess if Ror2 binding to and phosphorylation of 14-3-3β is direct we performed in vitro experiments with recombinant purified proteins. For the binding experiment, the GST fusion of the cytosolic domain of human Ror2 (GST-Ror2c) was expressed in bacterial cells, precipitated on glutathione sepharose and incubated with in vitro translated 14-3-3 β. As shown in FIG. 6A, 14-3-3 β bound to GST-Ror2c, but not to GST alone, indicating that 14-3-3 β binds directly to the cytosolic domain of Ror2. Since in vitro translated 14-3-3 β contained Expressway™ protein synthesis buffer that was incompatible with the kinase assay, we purchased purified recombinant GST-tagged 14-3-3β and performed in vitro kinase assay with purified recombinant GST-Ror2c (Invitrogen). As shown in FIG. 6B, Ror2c phosphorylated both 14-3-3 β and itself confirming that 14-3-3 β is a direct substrate for the Ror2 tyrosine kinase.

### Example 5

### Ror2-Specific Antibody Dimerizes and Activates the Ror2 Receptor

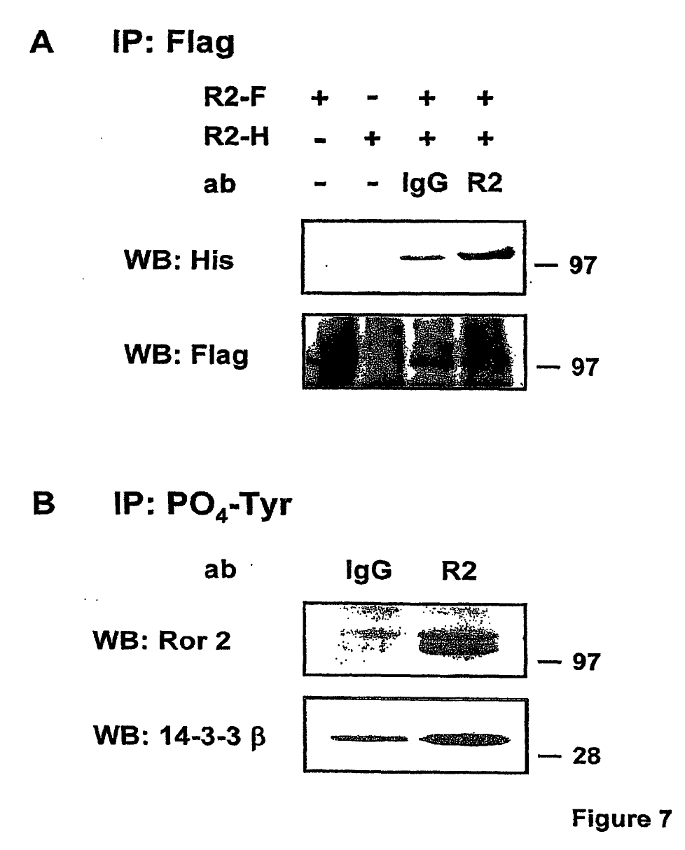

Several receptor tyrosine kinases have been shown to be dimerized and activated by antibodies (Spaargaren et al. J. Biol. Chem. 266, 1733-1739, 1991; Fuh et al. Science 256, 1677-1680, 1992; each of which is incorporated herein by reference). We therefore tested an Ror2-specific antibody raised against the entire extracellular domain of human Ror2 for its ability to dimerize and activate the Ror2 receptor tyrosine kinase. To assess dimerization, Flag-tagged and His-tagged Ror2 receptor constructs were expressed in U2OS cells and cells were treated for 1 h at 37 C with Ror2-specific goat polyclonal IgG (raised against the extracellular domain of human Ror2, R&D Systems, AF2064) or with non-specific goat IgG control (R&D Systems). Upon incubation, total cellular proteins were extracted, precipitated on anti-Flag affinity agarose and subjected to immunoblotting with anti-His antibody. As shown in the top panel of FIG. 7A, under control conditions of non-specific IgG treatment, there was some association between His-tagged and Flag-tagged Ror2 receptors indicating that Ror2 forms homodimers upon over-expression in U2OS cells. This homodimer formation was strongly enhanced upon treatment with Ror2 antibody confirming that the antibody can dimerize the Ror2 receptor. The experimental design was validated by the fact that anti-Flag antibody failed to immunoprecipitate Ror2-His in absence of Ror2-Flag and that anti-His antibody did not recognize the Ror2-Flag protein (FIG. 7A, top panel). Equal levels of Ror2-Flag precipitation were verified by immunoblotting with anti-Flag antibody (FIG. 7A, bottom panel).

To address if the antibody activated the Ror2 tyrosine kinase, we treated U2OS cells with the Ror2-specific antibody or the IgG control for 1 h at 37° C., isolated whole-cell protein extracts and precipitated all tyrosine phosphorylated proteins on phosphotyrosine antibody. FIG. 7B illustrates that treatment with anti-Ror2 antibody resulted in significant autophosphorylation of the Ror2 kinase as well as in phosphorylation of its substrate, 14-3-3β protein. These data provide strong evidence that anti-Ror2 antibody dimerizes and activates the Ror2 tyrosine kinase receptor.

### Example 6

### Ror2-Specific Activating Antibody Promotes hMSC Mineralization

We next asked if antibody-induced dimerization and activation of Ror2 will have functional consequences on osteogenic differentiation of hMSC. Since hMSC do not express Ror2 unless differentiated towards osteogenic phenotype (Billiard and Bodine, U.S. patent application, U.S. Ser. No. 10/823,998, filed Apr. 14, 2004; Billiard et al. Mol Endo 19, 90-101, 2005; each of which is incorporated herein by reference), we induced Ror2 expression by treatment with osteogenic cocktail (MSCGM supplemented with 0.05 mM ascorbic acid, 10 mM β-glycerophosphate, and 100 nM dex) and added increasing amounts of Ror2-specific goat IgG or non-specific goat IgG. After 9 days of incubation, the degree of mineralized matrix formation was assessed with alizarin red-S histochemical staining. As shown in FIG. 8, anti-Ror2 antibody dose-dependently increased the extent of calcified matrix formation in hMSC. Non-specific goat IgG had no effect on matrix mineralization at all concentrations tested, except a slight inhibition observed at the highest dose of 100 μg/ml. For clarity, only one dose of non-specific IgG (50 μg/ml) is shown in FIG. 8. A goat polyclonal antibody raised against the entire extracellular domain of human Ror1 (R&D Systems, AF2000) was also without effect (FIG. 8). The anti-Ror2 antibody effect was mediated through the Ror2 receptor, since it disappeared when the Ror2 expression in hMSC was inhibited by Ror2-specific shRNA (FIG. 9A). Furthermore, anti-Ror2 antibody induced calcified matrix formation even in absence of dex if the cells were induced to express Ror2 through adenovirus infection (FIG. 9B). Thus Ror2-specific antibody can dimerize and activate Ror2 receptor and promote Ror2-mediated calcified matrix formation in mesenchymal stem cells, suggesting that an activating Ror2 antibody may provide effective therapy for osteoporosis and other bone diseases.

### Example 7

### Inhibition of 14-3-3β Enhances hMSC Mineralization

To test if 14-3-3β B plays a role in osteogenic differentiation, we inhibited 14-3-3β expression in absence and presence of Ror2 over-expression. To this end, hMSC were infected with the 14-3-3β-specific shRNA, which indeed strongly down-regulated the endogenous protein expression when compared to scrambled control shRNA (FIG. 10A). When hMSC were infected with two control viruses, containing scrambled shRNA and β-galactosidase, we observed a low extent of matrix mineralization (FIG. 10B), suggesting that dual infection by itself mediates mild osteogenesis in hMSC cultures. As observed before (Billiard et al, U.S. patent application U.S. Ser. No. 10/823,998, filed Apr. 14, 2004), infection with Ror2 adenovirus strongly promoted the formation of mineralized matrix. To our surprise, down-regulation of 14-3-3β also greatly increased the extent of mineralization (FIG. 10B). Together, Ror2 over-expression and 14-3-3β inhibition induced stronger matrix mineralization than did either one alone (FIG. 10B). This is the first evidence to suggest that 14-3-3β scaffold protein exerts inhibitory effects on osteogenic differentiation of hMSC.

### Example 8

### Antibody-Induced Ror2 Activation and 14-3-3β Inhibition Promote New Bone Formation in Ex Vivo Calvarial Cultures

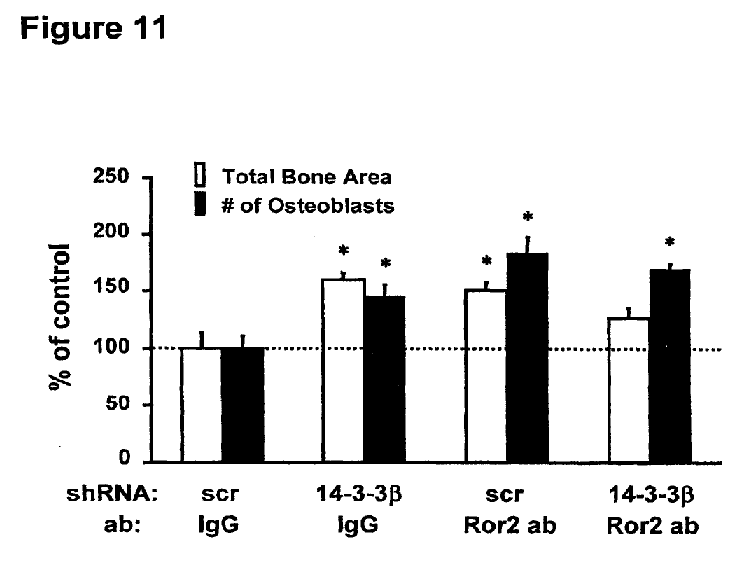

We next tested if in vitro effects of Ror2 activation and 14-3-3β inhibition translate into increased bone formation in ex vivo organ cultures. Calvarial bones of 4 days old mouse littermates were infected with adenoviruses containing scrambled shRNA or 14-3-3β-specific shRNA at 5×10⁷ viral particles/ml; and 48 h later were treated with 12 μg/ml of anti-Ror2 antibody or non-specific IgG in presence of 15 μg/ml calcein. After 7 days of culture with adenoviruses and antibodies, the bones were stained with hematoxylin-eosin and consistent 300 μm stretches (450 μm away from frontal sutures) were subjected to histomorphometric analysis using Bioquant-NOVA MR 5.50.8 (Nashville, Tenn.). Under control conditions of scrambled shRNA infection and IgG treatment, 600 μm length of calvaria contained 9394±1333 μm² of bone area and 39.5±7.4 osteoblasts. Inhibition of 14-3-3β by the specific shRNA caused 60% increase in the total bone area and 50% increase in the osteoblast number, indicating that 14-3-3β protein inhibits osteoblast counts and/or activity (FIG. 11). Treatment of calvarial bones with anti-Ror2 antibody resulted in 85% increase in osteoblast number and 50% increase in total bone area, once again suggesting that an activating Ror2 antibody may provide effective therapy for osteoporosis and other bone diseases. However, combining the 14-3-3β shRNA infection with the Ror2 antibody treatment did not produce an additive effect, resulting in even a slightly smaller response compared to either treatment alone (FIG. 11). Since in our experience the observed increases of 50-80% do not reach saturation in this assay system, it is tempting to speculate that both Ror2 and 14-3-3β are in the same pathway controlling osteoblast differentiation and/or function.

### Example 9

### Developing a High Throughput, High Sensitivity Assay for Ror2 Activity

As shown in FIG. 12A, the assay utilizes the well-characterized TrkB receptor signaling pathway. The TrkB receptor is activated by ligand-induced homo-dimerization that causes phosphorylation of Erk and stimulation of the cAMP response element (CRE) in the promoter of target genes. We generated a chimeric receptor consisting of the extracellular domain of Ror2 (aa 1-407) fused to the transmembrane and intracellular domains of TrkB (aa 432-822). We hypothesized that when using this chimera, agents that cause Ror2 dimerization will activate TrkB signaling pathway and result in increases in the CRE promoter activity. To test this hypothesis, the chimeric receptor was stably transfected into HEK293A cells over-expressing the CRE-luciferase plasmid (HEK-CRE) obtained from Dr. Seongeun Cho (Wyeth Research, Princeton, N.J.). The Ror2-TrkB chimera in pcDNA3.1(+)-hygro was electroporated into HEK-CRE cells using ECM 600 electroporator (BTX, San Diego, Calif.) and the cells were grown with 350 μg/ml hygromycin until isolated colonies of hygromycin-resistant cells were formed. Colonies were trypsinized and transferred one per well onto 96-well plates. Colonies were grown at 37 C with 350 μg/ml hygromycin and levels of Ror2-TrkB expression were assessed by western immunoblotting and immunocytochemistry. The HEK-CRE cells that expressed Ror2-TrkB chimera were treated with the anti-Ror2 antibody that has been previously shown to dimerize Ror2 (see Example 5). As shown in FIG. 12, Ror2-specific antibody caused a robust dose-dependent increase in the observed luciferase activity when compared to cells treated with non-specific IgG. Thus we have developed a rapid, high throughput and highly sensitive assay for measuring the ability of agents (including, but not limited to, small molecules, peptides, proteins, or antibodies) to induce Ror2 dimerization and activation.

What is claimed is:

1. A method of treating or preventing a bone-related disorder comprising administering to a subject with a bone-related disorder a therapeutically effective amount of an agent capable of activating Ror2 protein.

2. The method of claim 1, wherein the bone-related disorder is associated with bone loss.

3. The method of claim 1, wherein the disorder is selected from the group consisting of osteoporosis, bone cancer, arthritis, rickets, bone fracture, periodontal disease, bone segmental defects, osteolytic bone disease, primary and secondary hyperparathyroidism, Paget's disease, osteomalacia, and hyperostosis.

4. The method of claim 1, wherein the subject is human.

5. The method of claim 1, wherein the agent causes the dimerization of Ror2 protein.

6. The method of claim 1, wherein the agent is a small molecule.

7. The method of claim 1, wherein the agent is a protein.

8. The method of claim 1, wherein the agent comprises an antibody directed to Ror2 protein.

9. The method of claim 1, wherein the agent comprises a monoclonal antibody directed to Ror2 protein.

10. The method of claim 1, wherein the agent is a human or humanized monoclonal antibody directed to Ror2.

11. The method of claim 8, wherein the antibody is of the IgG isotype.

12. The method of claim 1, wherein the agent comprises an antibody fragment directed to Ror2 protein.

13. The method of claim 1, wherein the agent comprises an F_{ab} fragment directed to Ror2 protein.

14. The method of claim 1, wherein the agent comprises at least two antibody fragments directed to Ror2 protein, wherein the antibody fragments are linked together covalently.

15. The method of claim 1, wherein the agent is administered parenterally.

16. The method of claim 1, wherein the agent is administered intravenously.

17. The method of claim 1, wherein the agent is administered orally.

18. A method of increasing osteoblast differentiation comprising contacting cells expressing Ror2 with an agent capable of activating the Ror2.

19. The method of claim 18, wherein the agent is a protein.

20. The method of claim 18, wherein the agent is a small molecule.

21. The method of claim 18, wherein the agent is an antibody.

22. The method of claim 21, wherein the agent is an antibody directed to Ror2 protein.

23. The method of claim 18, wherein the agent causes the dimerization of Ror2 protein.

24. The method of claim 18, wherein the agent increases the phosphorylation of 14-3-3β by Ror2 protein.

25. The method of claim 18, wherein the cells are human cells.

26. The method of claim 18, wherein the cells are stem cells.

27. The method of claim 18, wherein the cells are mesenchymal stem cells.

28. The method of claim 18, wherein the step of contacting is performed ex vivo.

29. The method of claim 18, wherein the step of contacting is performed in vivo.

30. A method of inhibiting adipogenic differentiation comprising contacting a cell with an agent that increases the expression or activity of Ror2 protein.

31. A method of screening for an agent that increases Ror2 activity comprising steps of:
contacting cells expressing Ror2 protein with a test agent; and
determining whether Ror2 activity is increased.

32. The method of claim 31, wherein Ror2 is the cellular domain of Ror2.

33. The method of claim 31, wherein Ror2 is the kinase domain of Ror2.

34. The method of claim 31, wherein the step of determining comprises assessing the kinase activity of Ror2 protein.

35. The method of claim 34, wherein the step of assessing kinase activity of Ror2 protein comprises assessing the phosphorylation status of Ror2 protein.

36. The method of claim 31, wherein the step of determining comprises assessing expression levels of Ror2 protein or polynucleotide.

37. The method of claim 31, wherein the step of determining comprises assessing the phosphorylation status of 14-3-3β protein.

38. The method of claim 31, wherein the step of determining comprises determining the level of mineralized matrix formation.

39. An agent identified by the method of claim 31.

40. An antibody directed to Ror2, whereby the antibody causes the activation of Ror2 protein.

41. The antibody of claim 40, wherein the antibody causes the dimerization of Ror2 protein.

42. The antibody of claim 40, wherein the antibody is polyclonal.

43. The antibody of claim 40, wherein the antibody is monoclonal.

44. The antibody of claim 40, wherein the antibody is human.

45. The antibody of claim 40, wherein the antibody is humanized.

46. The antibody of claim 40, wherein the antibody is of the IgG isotype.

47. The antibody of claim 40, wherein the antibody comprises an antibody fragment.

48. The antibody of claim 40, wherein the antibody fragment is an Fab fragment.

49. The antibody of claim 40, wherein the antibody has at least two binding sites for Ror2 protein.

50. The antibody of claim 40, wherein the antibody has exactly two binding site for Ror2 protein.

51. A method of treating or preventing a bone-related disorder comprising administering to a subject with a bone-related disorder a therapeutically effective amount of an agent capable of inhibiting 14-3-3β activity.

52. The method of claim 51, wherein the agent down-regulates 14-3-3β expression.

53. The method of claim 51, wherein the agent is a 14-3-3β-specific siRNA or shRNA.

54. A method of identifying agents that promote the dimerization of Ror2 protein, the method comprising steps of:
providing a cell expressing a chimeric receptor including the extracellular domain of Ror2 and the intracellular domain of TrkB, wherein the cell comprises a reporter gene construct operably linked to a cAMP response element (CRE) promoter;
contacting the cell with a test agent; and
determining the level of expression of the reporter gene in the cell.

55. The method of claim 54, wherein the reporter gene is luciferase.

56. The method of claim 54, wherein the cell comprises a plasmid including a gene encoding luciferase operably linked to the CRE promoter.

57. A protein of amino acid sequence:
(SEQ ID NO: 4)
MARGSALPRRPLLCIPAVWAAAALLLSVSRTSGEVEVLDPNDPLGPLDGQ
DGPIPTLKGYFLNFLEPVNNITIVQGQTAILHCKVAGNPPPNVRWLKNDA
PVVQEPRRIIIRKTEYGSRLRIQDLDTTDTGYYQCVATNGMKTITATGVL
FVRLGPTHSPNHNFQDDYHEDGFCQPYRGIACARFIGNRTIYVDSLQMQG
EIENRITAAFTMIGTSTHLSDQCSQFAIPSFCHFVFPLCDARSRAPKPRE
LCRDECEVLESDLCRQEYTIARSNPLILMRLQLPKCEALPMPESPDAANC
MRIGIPAERLGRYHQCYNGSGMDYRGTASTTKSGHQCQPWALQHPHSHHL
SSTDFPELGGGHAYCRNPGGQMEGPWCFTQNKNVRMELCDVPSCSPRDSS
KMGILYlsvyavvviasvvgfcllvmlfllklarhskfgmkgpasvisnd
ddsasplhhisngsntpssseggpdaviigmtkipvienpqyfgitnsql
kpdtfvqhikrhnivlkrelgegafgkvflaecynlcpeqdkilvavktl
kdasdnarkdfhreaelltnlqhehivkfygvcvegdplimvfeymkhgd
lnkflrahgpdavlmaegnppteltqsqmlhiaqqiaagmvylasqhfvh
rdlatrnclvgenllvkigdfgmsrdvystdyyrvgghtmlpirwmppes
imyrkfttesdvwslgvvlweiftygkqpwyqlsnneviecitqgrvlqr
prtcpqevyelmlgcwqrephmrknikgihtlqnlakaspvyldilg;
or an amino acid sequence at least 90% homologous to SEQ ID NO: 4.

58. The protein of claim 57, wherein the amino acid sequence is:
(SEQ ID NO: 4)
MARGSALPRRPLLCIPAVWAAAALLLSVSRTSGEVEVLDPNDPLGPLDGQ
DGPIPTLKGYFLNFLEPVNNITIVQGQTAILHCKVAGNPPPNVRWLKNDA
PVVQEPRRIIIRKTEYGSRLRIQDLDTTDTGYYQCVATNGMKTITATGVL
FVRLGPTHSPNHNFQDDYHEDGFCQPYRGIACARFIGNRTIYVDSLQMQG
EIENRITAAFTMIGTSTHLSDQCSQFAIPSFCHFVFPLCDARSRAPKPRE
LCRDECEVLESDLCRQEYTIARSNPLILMRLQLPKCEALPMPESPDAANC
MRIGIPAERLGRYHQCYNGSGMDYRGTASTTKSGHQCQPWALQHPHSHHL
SSTDFPELGGGHAYCRNPGGQMEGPWCFTQNKNVRMELCDVPSCSPRDSS
KMGILYlsvyavvviasvvgfcllvmlfllklarhskfgmkgpasvisnd
ddsasplhhisngsntpssseggpdaviigmtkipvienpqyfgitnsql
kpdtfvqhikrhnivlkrelgegafgkvflaecynlcpeqdkilvavktl
kdasdnarkdfhreaelltnlqhehivkfygvcvegdplimvfeymkhgd
lnkflrahgpdavlmaegnppteltqsqmlhiaqqiaagmvylasqhfvh
rdlatrnclvgenllvkigdfgmsrdvystdyyrvgghtmlpirwmppes
imyrkfttesdvwslgvvlweiftygkqpwyqlsnneviecitqgrvlqr
prtcpqevyelmlgcwqrephmrknikgihtllqnlakaspvyldilg;
or an amino acid sequence at least 95% homologous to SEQ ID NO: 4.

59. The protein of claim 57, wherein the amino acid sequence is:
(SEQ ID NO: 4)
MARGSALPRRPLLCIPAVWAAAALLLSVSRTSGEVEVLDPNDPLGPLDGQ
DGPIPTLKGYFLNFLEPVNNITIVQGQTAILHCKVAGNPPPNVRWLKNDA
PVVQEPRRIIIRKTEYGSRLRIQDLDTTDTGYYQCVATNGMKTITATGVL
FVRLGPTHSPNHNFQDDYHEDGFCQPYRGIACARFIGNRTIYVDSLQMQG
EIENRITAAFTMIGTSTHLSDQCSQFAIPSFCHFVFPLCDARSRAPKPRE
LCRDECEVLESDLCRQEYTIARSNPLILMRLQLPKCEALPMPESPDAANC
MRIGIPAERLGRYHQCYNGSGMDYRGTASTTKSGHQCQPWALQHPHSHHL
SSTDFPELGGGHAYCRNPGGQMEGPWCFTQNKNVRMELCDVPSCSPRDSS
KMGILYlsvyavvviasvvgfcllvmlfllklarhskfgmkgpasvisnd
ddsasplhhisngsntpssseggpdaviigmtkipvienpqyfgitnsql
kpdtfvqhikrhnivlkrelgegafgkvflaecynlcpeqdkilvavktl
kdasdnarkdfhreaelltnlqhehivkfygvcvegdplimvfeymkhgd
lnkflrahgpdavlmaegnppteltqsqmlhiaqqiaagmvylasqhfvh
rdlatrnclvgenllvkigdfgmsrdvystdyyrvgghtmlpirwmppes
imyrkfttesdvwslgvvlweiftygkqpwyqlsnneviecitqgrvlqr
prtcpqevyelmlgcwqrephmrknikgihtllqnlakaspvyldilg.


SEQUENCE LISTING (4 sequences)

SEQ ID NO: 1 - DNA, 18 bp, Artificial Sequence
  Forward primer for human cyclophilin B
caccaacggctcccagtt

SEQ ID NO: 2 - DNA, 21 bp, Artificial Sequence
  Reverse primer for human cyclophilin B
aacaccacatgcttgccatct

SEQ ID NO: 3 - DNA, 27 bp, Artificial Sequence
  Probe for human cyclophilin B
ttcatcacgacagtcaagacagcctgg

SEQ ID NO: 4 - protein, 798 aa, Artificial Sequence
  Ror2/Trk B chimeric receptor
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRAPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG